(19) United States
(12) Patent Application Publication
(10) Pub. No.: US 2024/0156908 A1
(43) Pub. Date: May 16, 2024

(54) USE OF sEphB4-HSA FUSION PROTEIN AS A FIRST-LINE THERAPY IN CANCER TREATMENT

(71) Applicant: Valery Krasnoperov, Pasadena, CA (US)

(72) Inventor: Valery Krasnoperov, Pasadena, CA (US)

(21) Appl. No.: 18/282,538

(22) PCT Filed: Mar. 18, 2022
(86) PCT No.: PCT/US22/20878

§ 371 Date: Sep. 17, 2023

Related U.S. Application Data
(60) Provisional application No. 63/162,691, filed on Mar. 18, 2021.

(51) Int. Cl. A61K 38/17; A61K 31/337; A61K 39/00; A61P 35/00; C07K 16/28

(52) U.S. Cl. CPC A61K 38/1793; A61K 31/337; A61P 35/00; C07K 16/2863; A61K 2039/505

(57) ABSTRACT

Disclosed herein are methods using sEphB4-HSA an effective first-line therapy for cancers where current therapies are ineffective, result in relapse, or are not even considered for use due to the type of cancer and related tumors.

### RELATED APPLICATIONS

The application claims the benefit of, and priority to, U.S. Provisional Application No. 63/162,691, filed Mar. 18, 2021, the contents of which are hereby incorporated by reference in their entirety.

### TECHNICAL FIELD

The present disclosure provides, in part, compositions and methods comprising soluble Ephrin-HSA fusion proteins and uses thereof, including methods of treatment for cancer.

### SEQUENCE LISTING

The contents of the text file submitted electronically herewith are incorporated herein by reference in their entirety: A computer readable format copy of the Sequence Listing (Filename: “VAS-002PC_ST25.txt”; Date recorded: Mar. 17, 2022; File size: 14,127 bytes).

### BACKGROUND

Today, cancer remains a major cause of death worldwide despite the numerous advanced diagnostic and therapeutic methods that have been developed. In humans, cancers become established after a primary genetic event by a number of mechanisms that include, but are not limited to, increased cellular metabolism and growth rate, stimulation of angiogenesis thereby increasing blood supply to the tumor, and dysregulation of signaling pathways and tumor suppressors. Curative treatment protocols in clinical oncology remain reliant upon a combination of surgical resection, ionizing radiation, and cytotoxic chemotherapy. The major barrier to successful treatment and prevention of cancer lies in the fact that many cancers still fail to respond to the current chemotherapeutic and immunotherapy intervention, and many individuals suffer a recurrence or death, even after aggressive therapy. Moreover, tumors may become resistant to anti-cancer drugs by a number of mechanisms that include, but are not limited to, expulsion of the drug from the cell, occurrence of mutations that prevent binding of the drug to its target, and occurrence of additional mutations in genes and their protein products unrelated to the drug target. To address these shortcomings, there has been a trend in drug discovery to develop targeted therapies capable of modulating signaling axes dysregulated in cancers. There are now many FDA approved antibodies and small molecules that allow for therapeutic manipulation of a myriad of clinically relevant targets.

Eph (Erythropoietin Producing Hepatoma) receptor and ligand are part of the largest family of receptor tyrosine kinases (RTKs). The family is subdivided into class A and class B, based on sequence homology and binding affinity for two distinct types of membrane-anchored ephrin ligands. Each Eph receptor and ligand can bind to multiple ligands and receptors and certain receptors have been postulated as putative tumor suppressors and others as tumor promoters (Vaught et al. Breast Cancer Res, 10(6):217-224, 2008). EphrinB2 and its high affinity cognate receptor, EphB4, are transmembrane proteins that are induced in tumor vessels and regulate immune cell trafficking. Inhibition of the EphrinB2-EphB4 interaction has a direct inhibitory effect on tumor cell proliferation in vitro and ex-vivo.

### SUMMARY

In aspects, the present disclosure relates to a method for treating cancer comprising administering an effective amount of a polypeptide agent that inhibits EphB4 or EphrinB2-mediated functions to a patient in need thereof, wherein the polypeptide agent is used as a first-line therapy in the treatment.

In aspects, the present disclosure relates to use of a polypeptide agent that inhibits EphB4 or EphrinB2-mediated functions in preparing a medicament for use as a first-line therapy in treating a cancer. In embodiments, the cancer is selected from but not limited to, squamous cell carcinoma of the head and neck (HNSCC), hepatocellular carcinoma (HCC), Kras mutant non-small cell lung adenocarcinoma, and Kaposi sarcoma (KS).

In embodiments, the subject previously responded to treatment with an anti-cancer therapy, but, upon cessation of therapy, suffered relapse (hereinafter “a recurrent cancer”). In embodiments, the subject has resistant or refractory cancer. In embodiments, the cancer is refractory to platinum-based chemotherapy. In embodiments, the cancer is refractory to immunotherapy treatment. In embodiments, the cancer is refractory to treatment with a chemotherapeutic agent. In embodiments, the cancer is refractory to treatment using depleting antibodies to specific tumor antigens. In embodiments, the cancer is refractory to treatment using agonistic, antagonistic, or blocking antibodies to co-stimulatory or co-inhibitory molecules (immune checkpoints). In embodiments, the cancer is refractory to targeted treatment with an immunoconjugate, antibody-drug conjugate (ADC), or fusion molecule comprising a depleting antibody to specific tumor antigens tumor antigen and a cytotoxic agent. In embodiments, the cancer is refractory to targeted treatment with a small molecule kinase inhibitor. In embodiments, the cancer is refractory to treatment using surgery. In embodiments, the cancer is refractory to treatment using stem cell transplantation. In embodiments, the cancer is refractory to treatment using radiation. In embodiments, the cancer is refractory to combination therapy involving, for example, two or more of: immunotherapy treatment, treatment with a platinum based chemotherapeutic agent, treatment with a tumor antigen-specific, depleting antibody, treatment with a immunoconjugate, ADC, or fusion molecule comprising a tumor antigen-specific, depleting antibody and a cytotoxic agent, targeted treatment with a small molecule kinase inhibitor, treatment using surgery, treatment using stem cell transplantation, and treatment using radiation. In embodiments, the subject has a form of cancer for which it was determined that various anti-cancer therapies would not even be considered for use.

In embodiments, the use relates to methods of treating squamous cell carcinoma of the head and neck (HNSCC) in a subject, comprising administering to the subject a therapeutically effective amount of an sEphB4-HSA polypeptide as a first-line therapy. In embodiments, the HNSCC is refractory to treatment using platinum-based chemotherapy and/or radiation therapy. In embodiments, the HNSCC is refractory to treatment using a checkpoint inhibitor. In embodiments, the subject has relapsed HNSCC.

In embodiments, the use relates to methods of treating hepatocellular carcinoma (HCC) in a subject, comprising administering to the subject a therapeutically effective amount of an sEphB4-HSA polypeptide as a first-line therapy. In embodiments, the HCC is refractory to treatment using platinum-based chemotherapy and/or radiation therapy. In embodiments, the HCC is refractory to treatment using a checkpoint inhibitor. In embodiments, the subject has relapsed HCC.

In embodiments, the use relates to methods of treating Kras mutant non-small cell lung adenocarcinoma in a subject, comprising administering to the subject a therapeutically effective amount of an sEphB4-HSA polypeptide as a first-line therapy. In embodiments, the checkpoint inhibitor is a PD-1 inhibitor. In embodiments, the adenocarcinoma is refractory to treatment using platinum-based chemotherapy and/or radiation therapy. In embodiments, the adenocarcinoma is refractory to treatment using a checkpoint inhibitor. In embodiments, the subject has relapsed adenocarcinoma.

In embodiments, the use relates to methods of treating Kaposi sarcoma (KS) in a subject, comprising administering to the subject a therapeutically effective amount of an sEphB4-HSA polypeptide as a first-line therapy. In embodiments, the KS is refractory to treatment using platinum-based chemotherapy and/or radiation therapy. In embodiments, the KS is refractory to treatment using a checkpoint inhibitor. In embodiments, the subject has relapsed KS.

In embodiments, a soluble extracellular fragment of EphB4 fused to albumin (sEphB4-HSA) blocks interaction between Ephrin-B2 and EphB4, and blocks bidirectional signaling, thus promoting immune cell trafficking, and inducing an anti-tumor immune response in various cancers. As such, the present disclosure provided, in embodiments, an EphrinB2-EphB4 inhibitor, “sEphB4-HSA” (soluble extracellular fragment of EphB4 tyrosine kinase receptor fused to Human Serum Albumin) for the treatment of various cancers. sEphB4-HSA consists of the extracellular domain of human EphB4 receptor (sEphB4) fused in frame with human serum albumin (HSA). This fusion with HSA enhances the pharmacokinetics of sEphB4. sEphB4-HSA binds to the ligand of EphB4 tyrosine kinase receptor: the transmembrane protein Ephrin-B2. Through this binding, it blocks endogenous EphB tyrosine kinase receptors from interacting with EphrinB2. Data indicates that sEphB4-HSA reduces angiogenesis in tumors—thus starving tumors of blood and inhibits EphrinB2's ability to suppress recruitment of T cells to tumors—thus increasing T cell recruitment.

In embodiments, the polypeptide agent of sEphB4 for use in treating cancer further comprises use of an anti-EGFR antibody or antibody fragment thereof or a taxane. In aspects, a composition comprising a soluble EphB4-HSA fusion protein (sEphB4-HSA) and an anti-EGFR antibody, or fragment thereof for use in the treatment of cancer. In embodiments, the anti-EGFR antibody is cetuximab.

In embodiments, the present methods provide combination therapy with taxane, optionally paclitaxel (TAXOL) or docetaxel (TAXOTERE). In embodiments, the present methods provide combination therapy with anti-EGFR antibody, optionally cetuximab.

The data provided herein shows that sEphB4-HSA has the potential to be an effective first-line therapy for a number of cancers where current therapies are ineffective, result in relapse, or are not even considered for use due to the type of cancer and related tumors.

### BRIEF DESCRIPTION OF THE DRAWINGS

FIG. 1 depicts scans for a patient with tonsillar SCC treated weekly with 10 mg/kg sEphB4-HSA. Scans show partial response at week 8 of therapy and no sign of tumor at week 16.

FIG. 2 depicts scans for a patient with laryngeal SCC treated weekly with 10 mg/kg sEphB4-HSA. Scans show partial response at week 8 of therapy.

FIG. 3 depicts scans for a patient with tonsillar SCC (HPV-) treated weekly with 10 mg/kg sEphB4-HSA. Scans show partial response at week 8 of therapy and no sign of tumor at week 16. Patient came off study while responding and had stable disease.

FIG. 4 depicts scans for a patient with liver cancer (HCC) treated weekly with 10 mg/kg sEphB4-HSA. Scans show partial response at week 16 of therapy and no sign of tumor at week. Patient remains disease free off therapy for 8+months.

FIG. 5 depicts scans for a patient with liver cancer (HCC) treated weekly with 10 mg/kg sEphB4-HSA. Scans show partial response at week 16 of therapy. Patient remains on therapy with stable disease at 18+months from study entry.

FIG. 6 depicts scans for a patient with Kras mutant multifocal adenocarcinoma lung and brain metastasis treated weekly with 10 mg/kg sEphB4-HSA after progressing from previous treatment with cranial irradiation, and subsequently with carboplatin, paclitaxel, Avastin for 3 cycles. Patient has stable disease at 11+months from sEphB4-HSA therapy.

FIG. 7 depicts photos of a patient with Kaposi sarcoma (KS) treated weekly with 10 mg/kg sEphB4-HSA. The patient had complete resolution of the tumor and complete resolution of leg edema.

FIG. 8 depicts a graphical representation of the overall survival of advanced bladder cancer, newly diagnosed and treated with sEphB4-albumin fusion protein containing regimen.

FIG. 9 depicts a graphical representation of the neo-adjuvant therapy for muscle invasive bladder cancer, regimen containing sEphB4-albumin, without chemotherapy

FIG. 10 depicts the in vivo spontaneous breast tumor mouse model (MMTV-neu/Her2) response to sEphB4-HSA treatment. Mice were treated for five weeks at a dose of 7.5 mg/kg three times a week via IP injection. Tumor tissues were analyzed for Her2/ERBB2 total protein expression and phosphorylation. Lungs were analyzed for metastasis.

FIG. 11 depicts an exemplary ERBB2 exon 20 duplication response to sEphB4 therapy.

FIG. 12 depicts western blot analysis which illustrates how EphB4 binds and stabilizes EGFR. EphB4 binds EGFR; EphB4 knock-down lowers EGFR; EphB4 increases EGFR.

FIG. 13 depicts in vivo efficacy study for sEphB4-HSA and EGFR antibody. Control mice were treated with sEphB4+anti-EGFR antibody (Cetuximab) at 42 days, illustrating the synergistic effect.

FIG. 14 depicts in vivo tumor regression of cholangiocarcinoma in human patient (JG 64F). sEpHB4-HSA treatment was provided at 15 mg/kg Q2 wk for 15 months. Patient survival is 24+months since starting sEphB4-HSA treatment.

FIGS. 15A-15F depicts EphB4 expression confers a growth advantage to Kras mutated cells via a heatmap indicating the effect of mRNA-mediated knockdown of tyrosine kinases on cell lines (FIG. 15A), the effect of Kras depletion on 6 cell lines (FIG. 15B), the effect of EphB4 for Kas-mediated cell lines (FIG. 15C). EphB4 protein was enhanced in a dose-dependent manner by Kras (FIG. 15D), EphB4 and its ligand EphrinB2 were both increased in the tumors (FIG. 15E), and overexpression of EphB4 and EphrinB2 also observed in the tumors (FIG. 15F).

FIGS. 16A-16D depicts genetic ablation of EphB4 increases survival in Kras mutant mice where the mutant creates a premature stop codon in ephB4 gene after cre-mediated recombination (FIG. 16A), and EphB4 rearrangement (FIG. 16B). FIG. 16C shows K14KB4 (n=9) mice had significantly less tumor growth and extended survival. Carcinogenesis of lung adenocarcinoma was dramatically reduced in AdKPB4 (FIG. 16D).

FIGS. 17A-17D depicts the effects of knockdown of EphB4 attenuated AKT and ERK signaling in Kras-driven tumors. Signaling indicators, except for p-ERK1/2, detected in oral papillomas (FIG. 17A), but not in the tissues of EphB4 knockout mice for lung adenocarcinoma (FIG. 17B). FIG. 17C shows in situ and immunofluorescence staining showed overexpressed EphB4 mRNA and protein, respectively, in the tumor. FIG. 17D shows protein expression of Ad-Cre mice.

FIGS. 18A-18G depicts pharmacologic inhibition of EphB4 effectively inhibits Kras-driven tumorigenesis in vivo. FIG. 18A shows western blotting of the p-Tyr signal of EphB4 in sEphB4 treated tumors. FIG. 18B shows survival rates of both sEphB4 treated groups compared to that of control K14K mice. FIG. 18C shows the effect of prevention treatment of sEphB4 to K14KP on tumorigenesis and survival. FIG. 18D shows the effects of Taxol and sEphB4 combination treatment. FIG. 18E and FIG. 18F illustrates the effects of sEphB4 treatment on apoptosis and cell proliferation in tumors via TUNEL and Ki67 staining. FIG. 18G illustrates the abundance of P-AKT and P-S6 after EphB4 treatment.

FIGS. 19A-19F depicts the effects of EphB4 on 8-TrCP1-mediated ubiquitination and degradation of Kras. FIG. 19A shows knockdown of EphB4 by siRNA decreased endogenous Kras protein half-life. FIG. 19B shows Kras level in the tumor of K14K mice after sEphB4 treatment. FIG. 19C shows the effects of siRNA knockdown of EphB4 and EphB4 overexpression on Kras ubiquitination. FIG. 19D shows the effects of siRNA knockdown of 8-TrCP1 and 8-TrCP1 overexpression on Kras ubiquitination. FIG. 19E shows IP/western blot analysis of the effect of overexpressed EphB4 on 8-TrCP-mediated Kras poly-ubiquitination. FIG. 19F shows co-IP studies of protein-protein interactions between Kras and 8-TrCP1, EphB4 and 8-TrCP1, and EphB4 and Kras.

FIGS. 20A-20F depict the presence of a C-terminal EphB4 fragment modulates 8-TrCP1 ligase activity prompting Kras monoubiquitination at the Cys118 position. FIG. 20A shows bacterially purified His-tagged Kras proteins (wild type, WT or G12V mutant) subjected to in vitro ubiquitination using b-TrCP1-GFP immunoprecipitated from HEK293 cell lysates either in the presence or absence of in vitro transcribed and translated EphB4 C-ter fragment as indicated. Following 2 hrs of incubation, reactions were stopped by adding sample loading dye and subjected to immunoblotting using indicated antibodies. FIG. 20B shows a MS/MS spectrum of a peptide identifying ubiquitination of Cys118 in Kras. Peptides isolated on an in-gel digestion were resolved on a reverse phase column and collision-induced dissociation spectra were obtained using an Orbitrap XL mass spectrometer. FIG. 20C shows validation of the importance of C118 monoubiquitination, Cys118Ser mutants of Kras in G12D mutant background (named GC mutant) and subjected to in vitro ubiquitination along with WT and G12D (GD) mutant as described in panel (a) and processed/analyzed using immunoblotting using indicated antibodies. FIG. 20D shows the steady state levels of different KRAS (wild type, WT; G12D, GD; C118S, CS, and G12D±C118S, GC) mutants either in the presence and absence of EphB4 overexpression. Relative band intensities (arbitrary units) were quantified using Image J considering WT Kras level as ‘1’. Relative band intensities were also calculated for U-TrCP1 as above. FIG. 20E shows protein half-lives for WT, GD, CS and GC Kras mutants were calculated in the presence and absence of EphB4 by adding cycloheximide (CHX, 50 ug/ml). Samples were collected at the indicated time points, band intensities were calculated and plotted with time. FIG. 20F shows a hypothetical model indicating the importance of EphB4 in promoting C118 monoubiquitination necessary for mutant Kras hyperactivation. The targeting of EphB4 or genetic modification of C118 site to serine (S) may compromise mutant Kras oncogenic activity.

### DETAILED DESCRIPTION

In aspects, there is provided a method of treating a cancer comprising administering an effective amount of a polypeptide agent that inhibits EphB4 or EphrinB2, e.g. sEphB4 to a patient in need thereof, wherein the treatment is a first-line therapy.

In aspects, there is provided a method of treating a cancer comprising administering an effective amount of a polypeptide agent that inhibits EphB4 or EphrinB2, e.g. sEphB4 to a patient in need thereof, wherein the patient has not received treatment with another anti-cancer agent.

In aspects, there is provided a polypeptide agent that inhibits EphB4 or EphrinB2-mediated functions for use in the preparation of a medicament for use as a first-line therapy in treating a cancer. In aspects, there is provided a polypeptide agent that inhibits EphB4 or EphrinB2-mediated functions for use as a first-line therapy in treating a cancer.

### EphB4— EphrinB2 Inhibitors

The methods of the present disclosure include treating, reducing, or preventing primary tumor growth or formation of primary cancer, or metastasis of cancers by administering a polypeptide agent that inhibits EphB4 or EphrinB2-mediated functions, as first-line therapy.

Type one receptor tyrosine kinase EphB4 and membrane-localized ligand EphrinB2 induce bidirectional signaling (forward in receptor expressing cells, reverse signaling in ligand expressing cells). EphB4 belongs to the largest family of receptor tyrosine kinases and upon interaction with the EphrinB2 ligand has been reported to regulate neuronal migration, bone remodeling, angiogenesis, cancer progression, and metastasis (Pasquale E B, Cell, 133:38-52, 2008). EphB4 and EphrinB2 expression is downregulated in vast majority of adult normal tissues, even as early as postnatal development but EphB4 is over-expressed in multiple epithelial cancers including lung, bladder, head-neck, and pancreatic cancers (Ferguson B D, et el., Growth Factors, 32:202-6, 2014). Oncogenes including mutant Kras and loss of PTEN induce EphB4 expression. Expression of EphB4 correlates with stage, grade, and survival since knock down of EphB4 leads to cell death by apoptosis. The ligand EphrinB2's over-expression and correlation with poor outcome have been reported in several cancer types. ICT increases EphrinB2 in the tumor vessels (and tumor) and high EphrinB2 prevents immune cell recruitment and thus resistance to therapy.

Inhibition of the EphB4-EphrinB2 interaction has a direct inhibitory effect on tumor cell proliferation in vitro and ex-vivo. Polypeptide agents that inhibit EphB4 or EphrinB2-mediated functions have been previously described by the present inventors (see, e.g., U.S. Pat. Nos. 7,381,410; 7,862,816; 7,977,463; 8,063,183; 8,273,858; 8,975,377; 8,981,062; 9,533,026; each hereby incorporated by reference in their entirety for all purposes). sEphB4-HSA is a fully human fusion protein composed of soluble EphB4 extracellular domain fused at the C-terminus with albumin upon expression as a single seamless protein of 123.3 kDa. sEphB4-HSA specifically binds to EphrinB2. Preliminary studies of sEphB4-HSA in tumor models show increase in T and NK cell migration into tumor. This is accompanied by the induction of ICAM-1 in the tumor vessels. ICAM-1 is an integrin that promotes attachment of T and NK cells to the endothelium followed by transmigration of cells into the tumor. sEphB4-HSA also shows downregulation of P13K signaling by blocking EphB-EphrinB2 interaction in tumor cell and tumor vessels. sEphB4-HSA blocks the signaling and promote immune cell trafficking into the tumor and inhibit survival signal in tumor cells by downregulating P13K pathway.

Targeting of EphB4-EphrinB2 represent a therapeutic strategy that has survived the test of clinical trials. It has been shown to be safe in multiple clinical trials with minimal to no toxicity (A. El-Khoueiry B G, et al., Eur J Cancer, 69, 2016), likely due to low levels of expression in normal tissue. While direct evidence that implicates EphB4-EphrinB2 interaction in the cancer-related immune response is lacking, multiple reports have documented that Eph/ephrin gene family members modulate immune cell processes in inflammatory models, such as arteriosclerosis and wound healing (Braun J, et al., Arterioscler Thromb Vasc Biol, 31:297-305, 2011; Poitz D M, et al., Mol Immunol, 68:648-56, 2015; Yu G, et al., J Immunol, 171:106-14, 2003; Funk S D, et al., Arterioscler Thromb Vasc Biol, 32:686-95, 2012). Eph-ephrin interactions have also been reported to regulate monocyte adhesion to the blood vessel wall trans-endothelial migration, T cell chemotaxis, activation, proliferation and apoptosis, and mobilization of hematopoietic cells from bone marrow sinusoids.

In embodiments of the present disclosure, the polypeptide agent that inhibits EphB4 or EphrinB2-mediated functions is a monomeric ligand binding portion of the EphB4 protein or EphrinB2 protein, or an antibody that binds to and affects EphB4 or EphrinB2. In embodiments, the polypeptide agent is a soluble EphB4 (sEphB4) polypeptide that binds specifically to an EphrinB2 polypeptide and comprises an amino acid sequence of an extracellular domain of an EphB4 protein. In embodiments, the sEphB4 polypeptide comprises a globular domain of an EphB4 protein.

In embodiments, the sEphB4 polypeptide comprises a sequence selected from a sequence that is at least 90% identical to residues 1-522, at least 90% identical to residues 1-412, and at least 90% identical to residues 1-312 of the amino acid sequence of SEQ ID NO: 1. In embodiments, the sEphB4 polypeptide may comprise a sequence encompassing the globular (G) domain (amino acids 29-197 of SEQ ID NO; 1), and optionally additional domains, such as the cysteine-rich domain (amino acids 239-321 of SEQ ID NO: 1), the first fibronectin type 3 domain (amino acids 324-429 of SEQ ID NO: 1) and the second fibronectin type 3 domain (amino acids 434-526 of SEQ ID NO: 1). In embodiments, the sEphB4 polypeptide will comprise amino acids 1-537 of SEQ ID NO: 1. In embodiments, the sEphB4 polypeptide will comprise amino acids 1-427 of SEQ ID NO: 1. In embodiments, the sEphB4 polypeptide will comprise amino acids 1-326 of SEQ ID NO: 1. In embodiments, the sEphB4 polypeptide will comprise amino acids 1-197, 29-197, 1-312, 29-132, 1-321, 29-321, 1-326, 29-326, 1-412, 29-412, 1-427, 29-427, 1-429, 29-429, 1-526, 29-526, 1-537 and 29-537 of SEQ ID NO: 1. In embodiments, the sEphB4 polypeptide will comprise amino acids 16-197, 16-312, 16-321, 16-326, 16-412, 16-427, 16-429, 16-526 of SEQ ID NO: 1. In embodiments, a sEphB4 polypeptide may be one that comprises an amino acid sequence at least 90%, and optionally 95% or 99% identical to any of the preceding amino acid sequences while retaining EphrinB2 binding activity. In embodiments, any variations in the amino acid sequence from the sequence shown in SEQ ID NO: 1 are conservative changes or deletions of no more than 1, 2, 3, 4 or 5 amino acids, particularly in a surface loop region.

In embodiments, a soluble polypeptide may be prepared in a multimeric form, by, for example, expressing as an Fc fusion protein or fusion with another multimerization domain.

In embodiments, the sEphB4 polypeptide will further comprise an additional component that confers increased serum half-life while still retaining EphrinB2 binding activity. In embodiments, the sEphB4 polypeptides are monomeric and are covalently linked to one or more polyoxyaklylene groups (e.g., polyethylene, polypropylene). In embodiments, the sEphB4 polypeptide is covalently linked to a single polyethylene glycol (PEG) group (hereinafter “sEphB4-PEG”). In embodiments, the sEphB4 polypeptide is covalently linked to two, three, or more PEG groups.

In embodiments, the one or more PEG may have a molecular weight ranging from about 1 kDa to about 100 kDa, about 10 to about 60 kDa, and about 10 to about 40 kDa. The PEG group may be a linear PEG or a branched PEG. In embodiments, the soluble, monomeric sEphB4 conjugate comprises an sEphB4 polypeptide covalently linked to one PEG group of from about 10 to about 40 kDa (monoPEGylated EphB4), or from about 15 to 30 kDa, e.g. via an s-amino group of sEphB4 lysine or the N-terminal amino group. In embodiments, the sEphB4 is randomly PEGylated at one amino group out of the s-amino groups of sEphB4 lysine and the N-terminal amino group.

In embodiments, the sEphB4 polypeptide is stably associated with a second stabilizing polypeptide that confers improved half-life without substantially diminishing EphrinB2 binding. In embodiments, the stabilizing polypeptide is immunocompatible with human patients (or animal patients, where veterinary uses are contemplated) and will have little or no significant biological activity. In embodiments, the sEphB4 polypeptide is associated covalently or non-covalently with an albumin selected from a human serum albumin (HSA) (hereinafter “sEphB4-HSA”) and bovine serum albumin (BSA) (hereinafter “sEphB4-BSA”).

In embodiments, the covalent attachment may be achieved by expression of the sEphB4 polypeptide as a co-translational fusion with human serum albumin. The albumin sequence may be fused at the N-terminus, the C-terminus or at a non-disruptive internal position in the sEphB4 polypeptide. Exposed loops of the sEphB4 would be appropriate positions for insertion of an albumin sequence. Albumin may also be post-translationally attached to the sEphB4 polypeptide by, for example, chemical cross-linking. In embodiments, the sEphB4 polypeptide may also be stably associated with more than one albumin polypeptide.

In embodiments, the sEphB4-HSA fusion inhibits the interaction between EphrinB2 and EphB4, the clustering of EphrinB2 or EphB4, the phosphorylation of EphrinB2 or EphB4, or combinations thereof. In embodiments, the sEphB4-HSA fusion has enhanced in vivo stability relative to the unmodified wildtype polypeptide.

In embodiments, the sEphB4-HSA comprises residues 16-197 of SEQ ID NO: 1 directly fused to residues 25-609 of SEQ ID NO: 2. In embodiments, the sEphB4-HSA comprises residues 16-312 of SEQ ID NO: 1 directly fused to residues 25-609 of SEQ ID NO: 2. In embodiments, the sEphB4-HSA comprises residues 16-321 of SEQ ID NO: 1 directly fused to residues 25-609 of SEQ ID NO: 2. In embodiments, the sEphB4-HSA comprises residues 16-326 of SEQ ID NO: 1 directly fused to residues 25-609 of SEQ ID NO: 2. In embodiments, the sEphB4-HSA comprises residues 16-412 of SEQ ID NO: 1 directly fused to residues 25-609 of SEQ ID NO: 2. In embodiments, the sEphB4-HSA comprises residues 16-427 of SEQ ID NO: 1 directly fused to residues 25-609 of SEQ ID NO: 2. In embodiments, the sEphB4-HSA comprises residues 16-429 of SEQ ID NO: 1 directly fused to residues 25-609 of SEQ ID NO: 2. In embodiments, the sEphB4-HSA comprises residues 16-526 of SEQ ID NO: 1 directly fused to residues 25-609 of SEQ ID NO: 2. In embodiments, the sEphB4-HSA comprises residues 16-537 of SEQ ID NO: 1 directly fused to residues 25-609 of SEQ ID NO: 2.

### Head and Neck Squamous Cell Carcinoma (HNSCC)

In embodiments, the present disclosure relates to methods and use for the treatment of HNSCC, e.g. as a first-line therapy; and/or treatment of a subject previously responded to treatment with an anti-cancer therapy, but, upon cessation of therapy, suffered a recurrent cancer; and/or treatment of a subject resistant or refractory cancer.

Head and neck squamous cell carcinoma (HNSCC) accounts for almost 90% of cancers involving the upper aerodigestive tract (UADT). In the United States in 2005, cancers of the oral cavity, pharynx and larynx are expected to account for nearly 3% of incident cancers and 2% of cancer deaths. There are approximately 500,000 new cases diagnosed world-wide each year. Men are affected over two times more than women. Over half of these cancers involve the oral cavity. The rest are divided equally between larynx and pharynx. Numerous clinical trials are testing the benefits of immunotherapy in human cancer, including head and neck squamous cell carcinoma (HNSCC). The objective response rate is 6-20% (Szturz P, et al., BMC Med, 15:110, 2017; Ferris R L, et al., Oral Oncol, 81:45-51, 2018; Postow M A, et al., J Clin Oncol, 33:1974-82, 2015; Chow L Q M, et al., J Clin Oncol, 34:3838-45, 2016; Siu L L, et al., JAMA Oncol 2018) and the vast majority of patients demonstrate either innate or adaptive resistance to immunotherapy. Attempts at simply combining more immune checkpoint inhibitors have also proven disappointing due to increased toxicity to patients and lack of additional benefit (Clinical Trial No. NCT02205333). In orthotopic mouse models of HNSCC, we have recently demonstrated that tumor regrowth occurs even after combination treatment with anti-PDL1 antibody and radiation therapy (RT). Oweida A, et al., Clin Cancer Res, 2018; Messenheimer D J, et al., Clin Cancer Res, 23:6165-77, 2017).

Radiation therapy remains the standard of care treatment in the definitive management of patients with locally advanced HNSCCs and can act as an adjuvant for immunotherapy but there are some undesirable effects mounted in response to RT that in turn compromises the efficacy of immunotherapeutic agents. RT is unable to overcome the accumulation of immunosuppressive populations such as Tregs in the later (repair) phase. Therefore, finding other treatments that synergize with RT and counteract its negative effects is critical to overcome adverse side-effects, treatment resistance, and tumor regrowth.

Five-year survival rates for HNSCC are low and have not improved in several decades. Moreover, patients with this disease experience severe morbidity including disfigurement, speech, swallowing and breathing problems. Late stage of diagnosis and propensity to recur are challenges that thwart efforts to improve outcomes in these patients. Pembrolizumab is a potent and highly selective humanized monoclonal antibody (mAb) of the IgG4/kappa isotype designed to directly block the interaction between PD-1 and its ligands, PD-L1 and PD-L2. The Food and Drug Administration (FDA) approved pembrolizumab (KEYTRUDA®) on Aug. 5, 2016, for the treatment of some patients with an advanced form of head and neck cancer. The approval is for patients with recurrent or metastatic head and neck squamous cell carcinoma (HNSCC) that has continued to progress despite standard-of-care treatment with chemotherapy. According to the FDA approval summary, 28 patients (16%) experienced a tumor response following treatment with pembrolizumab. In 23 (82%) of those patients, the tumor response lasted for 6 months or longer, and several have lasted for more than 2 years. Patients with HNSCC whose tumors are positive for the human papillomavirus (HPV) typically have better outcomes after treatment with chemotherapy than patients whose tumors are HPV negative. According to the FDA approval summary, responses were seen in patients with HPV-positive tumors as well as in patients with HPV-negative tumors (24% and 16%, respectively).

Recurrent, locally advanced, or metastatic head and neck squamous cell carcinoma (HNSCC) is a life-threatening disease. Squamous cell cancers of the head and neck are heterogeneous tumors with prognosis depending on the site of origin. Two categories are oral cavity/pharyngeal and laryngeal. HPV causes oropharyngeal good risk group while HPV negative tumors carry poor risk. Over 48,000 new cases of oral cavity and pharyngeal cancer and over 13,000 cases of laryngeal cancer were diagnosed in the U.S. in 2016, with approximately 13,000 deaths due to these cancers. At the time of initial diagnosis, distant metastases are present in approximately 18% of patients with oral cavity/pharyngeal cancer and 19% of patients with laryngeal cancer. In addition, regional lymph node involvement (without distant metastasis) is present in approximately 47% of patients with oral cavity/pharyngeal cancer and 22% of patients with laryngeal cancer at the time of initial diagnosis; for patients with such locally advanced disease, 20%-30% will recur locally, while another 10%-15% can be expected to develop distant metastases. Median survival for patients with recurrent or metastatic HNSCC in most clinical series is 6-10 months.

Standard treatment for locally advanced HNSCC includes platinum-containing chemotherapy given in conjunction with radiation (e.g., as induction therapy, as concurrent therapy with radiation, or as part of adjuvant therapy with radiation following surgical resection). First-line chemotherapy for metastatic HNSCC consists of a multi-agent platinum-containing chemotherapy regimen, such as cisplatin or carboplatin plus 5-fluorouracil plus cetuximab. Most recently, PD1 antibody was approved for patients relapsed or refractory HNSCC after failure to platinum and cetuximab. Response rate for PD1 antibody alone is 16% and DOR ranged from 2.4+months to 27.7+months, indicative of durable response. There remains a need, however, for novel therapies for patients who either fail or experience unacceptable toxicity to chemotherapy, cetuximab and PD1 antibody.

Hepatocellular carcinoma (HCC)

In embodiments, the present disclosure relates to methods and use for the treatment of HCC, e.g. as a first-line therapy; and/or treatment of a subject previously responded to treatment with an anti-cancer therapy, but, upon cessation of therapy, suffered a recurrent cancer; and/or treatment of a subject resistant or refractory cancer.

Liver cancer accounts for more than 850,000 new cancer cases annually and approximately 90% of these are hepatocellular carcinoma (HCC). Chronic infection with hepatitis C virus (HCV) or hepatitis B virus (HBV) is the leading cause of HCC. HCC is the most frequent cancer in certain parts of the world, and the fifth most cancer common worldwide. Globally, it is the second leading cause of cancer death in men and the sixth leading cause of cancer death among women (see, e.g., Parkin D. M., Lancet Oncology, 2:533-43, 2001). Because HCC is often diagnosed late in the course of clinical manifestation, only 10-15% of patients are candidates for curative surgery. Multiple treatment modalities are available for local therapy and include surgery, chemical ablation, radio-ablation, chemoembolization with local disease control in substantial patient population. For the majority of HCC patients, systemic chemotherapies or supportive therapies are the mainstay treatment options. HCC in general is highly refractory to therapy and most chemotherapeutic agents show limited effectiveness and have not been able to improve patient survival (see, e.g., Gish R. G. et al., J. of Clinical Oncology 25:3069-75, 2007; Ramanathan R. K. et al., J. of Clinical Oncology 24:4010, 2006).

Sorafenib, a small molecule multi-kinase inhibitor, was the first systemic therapy approved for advanced hepatocellular carcinoma. In selected patients who tolerated sorafenib but progressed while on therapy, another multi-kinase inhibitor, regorafenib was approved which provides survival benefit over placebo control (10.6 months vs 7.8 months). Most recently, the combination of Atezolizumab and Bevacizumab showed superiority over sorafenib as first-line treatment. After a median follow up of 8.6 months the median overall survival was not reached in combination arm compared to 13.2 months in the sorafenib arm. Overall response rate was 27% in combination vs 12% in sorafenib arm.

Recent studies evaluating the Programmed Death 1 (PD-1) antibody nivolumab (OPDIVO®) showed response rates of around 10-20%. Response duration was 14-17+months for CR, <1-8+months for PR, and 1.5-17+months for stable disease (SD). Overall survival (OS) rate at 6 months is 72%. Nivolumab demonstrated a manageable AE profile and produced durable responses across all dose levels and HCC cohorts, with a favorable 6-month OS rate. PD-1 antibodies also have accelerated approval in second line therapy. Additional therapies are needed for patients who fail current approved therapies.

### Non-small Cell Lung Cancer (NSCLC)

In embodiments, the present disclosure relates to methods and use for the treatment of NSCLC, e.g. as a first-line therapy; and/or treatment of a subject previously responded to treatment with an anti-cancer therapy, but, upon cessation of therapy, suffered a recurrent cancer; and/or treatment of a subject resistant or refractory cancer.

NSCLC is the most common type of lung cancer. Squamous cell carcinoma, adenocarcinoma, and large cell carcinoma are all subtypes of NSCLC. NSCLC accounts for about 85% of all lung cancers. As a class, NSCLCs are relatively insensitive to chemotherapy, compared to small cell carcinoma. When possible, they are primarily treated by surgical resection with curative intent, although chemotherapy is increasingly being used both pre-operatively (neoadjuvant chemotherapy) and post-operatively (adjuvant chemotherapy). On Oct. 2, 2015, the FDA approved pembrolizumab for the treatment of metastatic non-small cell lung cancer (NSCLC) in patients whose tumors express PD-L1 and who have failed treatment with other chemotherapeutic agents. In October 2016, pembrolizumab became the first immunotherapy to be used first-line in the treatment of NSCLC if the cancer overexpresses PDL1 and the cancer has no mutations in EGFR or in ALK; if chemotherapy has already been administered, then pembrolizumab can be used as a second line treatment but if the cancer has EGFR or ALK mutations, agents targeting those mutations should be used first. Assessment of PDL1 must be conducted with a validated and approved companion diagnostic. In the Keynote-001 trial (NTC01295827), the efficacy and safety of programmed cell death 1 (PD-1) inhibition with pembrolizumab was assessed in patients with advanced non—small-cell lung cancer. Among all the patients, the objective response rate was 19.4%, and the median duration of response was 12.5 months. The median duration of progression-free survival was 3.7 months, and the median duration of overall survival was 12.0 months. PD-L1 expression in at least 50% of tumor cells was selected as the cutoff from the training group. Among patients with a proportion score of at least 50% in the validation group, the response rate was 45.2%. Among all the patients with a proportion score of at least 50%, median progression-free survival was 6.3 months; median overall survival was not reached. PD-L1 expression in at least 50% of tumor cells correlated with improved efficacy of pembrolizumab (Garon et al., N Engl J Med, 372:2018-2028, 2015). KRAS-mutant lung adenocarcinomas, accounting for 30% of non-small cell lung cancers, exhibit a high level of heterogeneity with emerging clinical significance. While tumor heterogeneity can be influenced by genetic and/or epigenetic alterations co-occurring with KRAS, heterogeneous tumor subsets can also be the product of differing cells-of-origin. Genetically engineered mouse models based on the spatial and temporal activation of oncogenic Kras have been instrumental in addressing these questions. Indeed, the observation that most tissues, with the exception of the lung, are resistant to KrasG 12 v oncogenic signals, highlights an exquisite cell type dependency of oncogenic Kras-driven transformation.

Kaposi sarcoma (KS)

In embodiments, the present disclosure relates to methods and use for the treatment of KS, e.g. as a first-line therapy; and/or treatment of a subject previously responded to treatment with an anti-cancer therapy, but, upon cessation of therapy, suffered a recurrent cancer; and/or treatment of a subject resistant or refractory cancer.

Kaposi sarcoma (KS) is a multifocal angioproliferative disorder of vascular endothelium, most associated with infection with the Kaposi-sarcoma associated herpes virus (KSHV), also known as human herpes virus-8 (HHV-8). KS is associated with a number of epidemiologic and pathophysiologic factors. KS is classified into four distinct clinical types: classic Mediterranean KS, African-endemic KS, immunosuppressive drug-related KS, and HIV-related KS. A rare disease before the era of HIV and AIDS, HIV-related KS is the most frequent malignancy in HIV infected patients. KS can affect many organs. KS manifests most frequently as a disease of the skin. In many advanced cases, KS involves organs such as the lungs, liver, or gastrointestinal tract. At this time, KS is incurable. Available therapies are for palliation. Systemic chemotherapy is generally used for patients with more advanced disease or evidence of rapid progression of disease. The major goals of treatment are symptom palliation, prevention of disease progression, and reduction of tumor burden to alleviate lymphedema, organ compromise, and psychological stress. The standard therapies for visceral or advanced cutaneous KS include cytotoxic chemotherapy such as liposomal anthracycline and paclitaxel. Liposomal doxorubicin has superior efficacy and favorable tolerability and toxicity compared to the combination of non-liposomal doxorubicin, vincristine, and bleomycin with overall response rates of 59% in HIV patients. In classical KS, response rates to liposomal doxorubicin can be higher. However, complete response rates are uncommon and there is no cure. At this point in time, no targeted therapy has been fully developed for KS.

In embodiments, the cancer is selected from, but not limited to, squamous cell carcinoma of the head and neck (HNSCC), hepatocellular carcinoma (HCC), Kras mutant non-small cell lung adenocarcinoma, and Kaposi sarcoma (KS).

In embodiments, the patient previously responded to treatment with an anti-cancer therapy, but, upon cessation of therapy, suffered relapse (hereinafter “a recurrent proliferative disease”).

In embodiments, the patient has resistant or refractory cancer. In embodiments, the cancer is refractory to immunotherapy treatment. In embodiments, the cancer is refractory to treatment with a chemotherapeutic agent. In embodiments, the cancer is refractory to treatment using depleting antibodies to specific tumor antigens. In embodiments, the cancer is refractory to treatment using agonistic, antagonistic, or blocking antibodies to co-stimulatory or co-inhibitory molecules (immune checkpoints). In embodiments, the cancer is refractory to targeted treatment with an immunoconjugate, antibody-drug conjugate (ADC), or fusion molecule comprising a depleting antibody to a specific tumor antigen and a cytotoxic agent. In embodiments, the cancer is refractory to targeted treatment with a small molecule kinase inhibitor. In embodiments, the cancer is refractory to combination therapy involving, for example, two or more of: immunotherapy treatment, treatment with a chemotherapeutic agent, treatment using depleting antibodies to specific tumor antigens, treatment using agonistic, antagonistic, or blocking antibodies to co-stimulatory or co-inhibitory molecules (immune checkpoints), treatment with a immunoconjugate, ADC, or fusion molecule comprising a depleting antibody to a specific tumor antigen and a cytotoxic agent, targeted treatment with a small molecule kinase inhibitor, treatment using surgery, treatment using stem cell transplantation, and treatment using radiation.

### Bladder Cancer

In embodiments, the present disclosure relates to methods and use for the treatment of bladder cancer, e.g. as a first-line therapy; and/or treatment of a subject previously responded to treatment with an anti-cancer therapy, but, upon cessation of therapy, suffered a recurrent cancer; and/or treatment of a subject resistant or refractory cancer.

In embodiments, the balder cancer is newly diagnosed locally advanced (beyond the bladder or draining system, ureter, renal pelvis) bladder and urothelial carcinoma. In embodiments, the bladder cancer patient has not received systemic therapy or is within 12 months of neo-adjuvant systemic chemotherapy.

In embodiments, the bladder cancer patient is ineligible to receive standard a cisplatinum containing regimen.

In embodiments, the bladder cancer tumor has a mutation in TP53, ARID-1, BAP-1, RAS, PBRM1, P13K, and/or PIK3CA. In embodiments, the bladder cancer tumor has a mutation in HER2 and/or EGFR2.

In embodiments, the bladder cancer is muscle invasive bladder cancer.

### Cholangiocarcinoma (CCA)

In embodiments, the present disclosure relates to methods and use for the treatment of CCA, e.g. as a first-line therapy; and/or treatment of a subject previously responded to treatment with an anti-cancer therapy, but, upon cessation of therapy, suffered a recurrent cancer; and/or treatment of a subject resistant or refractory cancer.

CCA constitutes a diverse group of malignancies emerging in the biliary tree. CCAs are divided into three subtypes depending on their anatomical site of origin: intrahepatic (iCCA), perihilar (pCCA) and distal (dCCA) CCA. Considered as an independent entity, mixed HCC—CCA tumors are a rare type of liver malignancy sharing features of both iCCA and HCC and presenting an aggressive disease course and poor prognosis. iCCAs arise above the second-order bile ducts, whereas the point of anatomical distinction between pCCA and dCCA is the insertion of the cystic duct. pCCA and dCCA can also be collectively referred to as ‘extrahepatic’ (eCCA). In the United States, pCCA is the single largest group, accounting for approximately 50-60% of all CCAs, followed by dCCA (20-30%) and iCCA (10-20%). CCA is the second most common primary hepatic malignancy after hepatocellular carcinoma (HCC), comprising approximately 15% of all primary liver tumors and 3% of gastrointestinal cancers. CCAs are usually asymptomatic in early stages and, therefore, often diagnosed when the disease is already in advanced stages, which highly compromises therapeutic options, resulting in a dismal prognosis. CCA is a rare cancer, but its incidence (0.3-6 per 100,000 inhabitants per year) and mortality (1-6 per 100,000 inhabitants per year, globally, not considering specific regions with incidence >6 per 100,000 habitants such as South Korea, China, and Thailand) have been increasing in the past few decades worldwide, representing a global health problem. Despite advances in CCA awareness, knowledge, diagnosis and therapies, patient prognosis has not improved substantially in the past decade, with 5-year survival (7-20%) and tumor recurrence rates after resection still disappointing. Large bile duct iCCA, similar to pCCA and dCCA, shows a high frequency of mutations in KRAS and/or TP53 genes. As discussed in Examples 1-2, tumors with mutation in TP53, ARID-1, BAP-1, RAS, PBRM1, P13K, PIK3CA, did not prevent response to sEphB4-HSA therapy. Further, co-administration of sEphB4-HSA+anti-EGFR (cetuximab) can have synergistic anti-tumor effects, especially in anti-EGFR-resistant cancers, for example, ostensibly due to factors such as HER2 overexpression, etc.

In embodiments, the CCA patient is resistant to cisplatinum and/or gemcitabine

Compositions for treating cholangiocarcinoma, among other cancer types, can include combination administration of a sEphB4-HSA fusion protein and an anti-EGFR antibody, or antibody fragment thereof (e.g. VHH, nanobody, scFv, etc.). In embodiments, the anti-EGFR antibody can be cetuximab, which is a monoclonal antibody (mAb). The antibody can be a humanized antibody, human antibody, chimeric antibody, among other antibody formats.

### HER2/EGFR2 Mutant Cancers

In embodiments, the present disclosure relates to methods and use for the treatment of HER2/EGFR2 mutant cancers, e.g. as a first-line therapy; and/or treatment of a subject previously responded to treatment with an anti-cancer therapy, but, upon cessation of therapy, suffered a recurrent cancer; and/or treatment of a subject resistant or refractory cancer.

In embodiments, the HER2/EGFR2 mutant cancer is a lung, head and neck or bladder cancer.

In embodiments, the HER2/EGFR2 mutant cancer patient has failed chemotherapy and/or kinase inhibitor treatment and/or Her2 antibody treatment (e.g. including ADCs).

In embodiments, the HER2/EGFR2 mutant cancer patient has an exon 20 p 772_A775 duplication Her2 mutation. In embodiments, the HER2/EGFR2 mutant cancer patient has concurrent mutations in RB1 Exon20 pL700X, TP53 Exon 4 p.S116fs.

In embodiments, the HER2/EGFR2 mutant cancer patient has an ERBB2 Exon 17 V659E mutation In embodiments, the HER2/EGFR2 mutant cancer patient has concurrent PIK3CA E 545K, TP53, exon 5, R158fs, and ATM G2891D NF1 E2143 mutations.

In embodiments, the HER2/EGFR2 mutant cancer patient has an ERBB2 mutation. In embodiments, the HER2/EGFR2 mutant cancer patient has a concurrent ATM, RICTOR, CCNE1, CDKN18, IRS2, PMS2, TERT, and TP53 mutation.

In embodiments, the EGFR mutant cancer shows a high EGFR level, e.g. head and neck cancer, lung cancer, colon cancer, and bladder cancer.

### KRAS Mutant Cancers

In embodiments, the present disclosure relates to methods and use for the treatment of kras mutant cancers, e.g. as a first-line therapy; and/or treatment of a subject previously responded to treatment with an anti-cancer therapy, but, upon cessation of therapy, suffered a recurrent cancer; and/or treatment of a subject resistant or refractory cancer.

In embodiments, the kras mutant cancer is selected from lung, colorectal and pancreatic cancer. In embodiments, the kras mutant cancer is selected from pancreatic ductal adenocarcinoma (PDAC) and non-small-cell lung cancer (NSCLC),

In embodiments, the kras mutation is selected from G12C, G12D and G12R.

In embodiments, the present methods cause a reduction or inhibition of Kras nucleic acid or protein levels. In embodiments, the present methods cause an increased proteolysis of Kras protein.

In embodiments, the present methods cause a reduction or inhibition of Kras-driven tumorigenesis, e.g. as compared to an untreated state.

### Pharmaceutical Compositions

In embodiments, the polypeptide therapeutic agents of the present disclosure are often administered as pharmaceutical compositions comprising an active therapeutic agent, i.e., and a variety of other pharmaceutically acceptable components. (See Remington's Pharmaceutical Science, 15th ed., Mack Publishing Company, Easton, Pa., 1980). The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In embodiments, pharmaceutical compositions for the treatment of primary or metastatic cancer can be administered by parenteral, topical, intravenous, intratumoral, oral, subcutaneous, intraarterial, intracranial, intraperitoneal, intranasal, or intramuscular means.

For parenteral administration, pharmaceutical compositions of the disclosure can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies and/or polypeptides can be administered in the form of a depot injection or implant preparation which can be formulated in such a manner as to permit a sustained release of the active ingredient. Typically, the pharmaceutical compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The polypeptide agents of this disclosure can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

In embodiments, methods of the present disclosure include administering to a patient in need of treatment a therapeutically effective amount or an effective dose of sEphB4-HSA polypeptide of the present disclosure. In embodiments, effective doses of the polypeptides of the present disclosure, e.g. for the treatment of primary or metastatic cancer, described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

In embodiments, the dosage may range from about 0.0001 to 100 mg/kg, and more usually 0.01 to 10 mg/kg, of the host body weight. For example, dosages can be 1 mg/kg body weight or 10 mg/kg body weight or within the range of 1-10 mg/kg. In embodiments, the dosage of the polypeptide administered to the patient is selected from about 0.5, of about 1.0, of about 1.5, of about 2.0, of about 2.5, of about 3.0, of about 3.5, of about 4.0, of about 4.5, of about 5.0, of about 6.0, of about 7.0, of about 8.0, of about 9.0, and of about 10.0 mg/kg. In embodiments, the treatment regime entails administration once per week. In embodiments, the treatment regime entails administration once per every two weeks or once a month or once every 3 to 6 months. Therapeutic entities of the present disclosure are usually administered on multiple occasions. Intervals between single dosages can be weekly, bi-weekly, monthly, or yearly. Intervals can also be irregular as indicated by measuring blood levels of the therapeutic entity in the patient. Alternatively, therapeutic entities of the present disclosure can be administered as a sustained release formulation, in which case less frequent administration is required. Dosage and frequency vary depending on the half-life of the polypeptide in the patient.

Toxicity of the polypeptides described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from the cell culture assays and animal studies can be used in formulating a dosage range that is not toxic for use in human. The dosage of the polypeptides described herein lies preferably within a range of circulating concentrations that include the effective dose with little or no toxicity. The dosage can vary within this range depending upon the dosage form employed and the route of administration utilized. The exact formulation, route of administration and dosage can be chosen by the subject physician in view of the patient's condition. (See, e.g., Fingl et al., 1975, In: The Pharmacological Basis of Therapeutics, Ch. 1).

In embodiments, the methods comprise one or more additional anti-cancer therapies selected from immunotherapy, chemotherapy, targeted treatment using depleting antibodies to specific tumor antigens, targeted treatment using agonistic, antagonistic, or blocking antibodies to co-stimulatory or co-inhibitory molecules (immune checkpoints), targeted treatment with an immunoconjugate, ADC, or fusion molecule comprising depleting antibodies to specific tumor antigens and a cytotoxic agent, small molecule kinase inhibitor targeted therapy, surgery, radiation therapy, and stem cell transplantation. The combination may be synergistic. The combination may increase the therapeutic index of the anti-cancer therapy.

In embodiments, the immunotherapy is selected from treatment using agonistic, antagonistic, or blocking antibodies to co-stimulatory or co-inhibitory molecules (immune checkpoints) such as PD-1, PD-L1, PD-L2, CTLA-4, OX-40, CD137, GITR, LAG3, TIM-3, and VISTA; treatment using bispecific T cell engaging antibodies (BITE®) such as blinatumomab: treatment involving administration of biological response modifiers such as IL-2, IL-12, IL-15, IL-21, GM-CSF and IFN-α, IFN-3 and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod. In embodiments, the immunotherapy is selected from treatment using agonistic, antagonistic, or blocking antibodies to co-stimulatory or co-inhibitory molecules; treatment using chimeric antigen receptor (CAR)-T cells; treatment using CAR-NK cells; and treatment using bispecific T cell engaging antibodies (BITE®). In embodiments, the immunotherapy is treatment using agonistic, antagonistic, or blocking antibodies to co-stimulatory or co-inhibitory molecules. In embodiments, the immunotherapy is treatment using chimeric antigen receptor (CAR)-T cells. In embodiments, the immunotherapy is treatment using CAR-NK cells. In embodiments, the immunotherapy is treatment using bispecific T cell engaging antibodies (BITE®).

Depending on the nature of the combinatory therapy, administration of the polypeptide therapeutic agents of the disclosure may be continued while the other therapy is being administered and/or thereafter. The polypeptide therapeutic agents may be administered prior to, concurrently with, or following the additional anti-cancer therapy, usually within at least about 1 week, at least about 5 days, at least about 3 days, at least about 1 day. The polypeptide therapeutic agents may be delivered in a single dose, or may be fractionated into multiple doses, e.g. delivered over a period of time, including daily, bidaily, semi-weekly, weekly, etc. The effective dose will vary with the route of administration, the specific agent, the dose of anti-cancer agent, and the like, and may be determined empirically by one of skill in the art.

The following examples are provided to describe the disclosure in further detail.

### EXAMPLES

### Example 1: Phase I/II trial of sEphB4-HSA Monotherapy in Head & Neck SCC

In this study, 18 HNSCC patients were accrued, including 7 during dose escalation cohort and 11 in the expansion cohort. 8 patients were HPV negative, and 10 patients were HPV positive. One patient with adenocystic carcinoma of the parotid gland was excluded from analysis. One patient withdrew within the first three weeks of therapy.

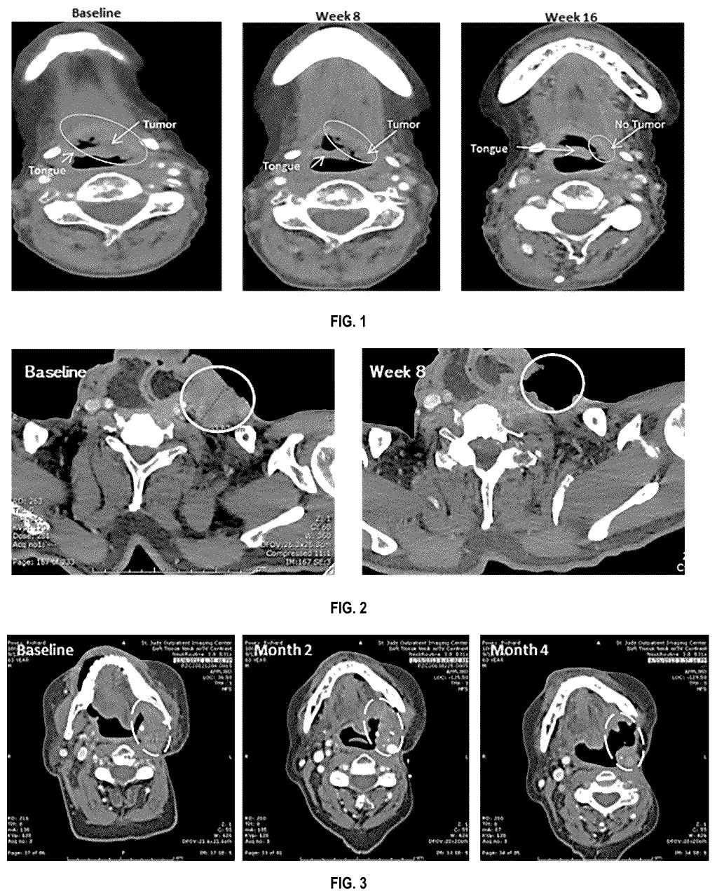

16 patients were evaluated for response. Patients received intravenous sEphB4-HSA 10 mg/kg every week. 15 patients had previously received curative radiation and chemotherapy. Ten patients had surgery either at diagnosis or at relapse. All patients received chemotherapy for relapsed HNSCC. Prior systemic therapy ranged from 2 to 6 different regimens. In addition, 12 patients previously received cetuximab and one patient received PD1 antibody. The results are presented in Table 1 below and in FIG. 1, FIG. 2, and FIG. 3.

TABLE 1
Patient response; CR (complete response), PR (partial response), MR
(mixed response,) SD (stable disease), PD (progressive disease).

| | | HPV | HPV |
|---|---|---|---|
| Response | ALL | Negative | Positive |
| Patients on Study | 18 | 8 | 10 |
| CR/PR/MR | 2 (PR) 1 (MR) | 3 | 0 |
| Tumor regression | 2 | 0 | 2 |
| SD (4 months or more) | 5 | 2 | 3 |
| PD | 6 | 3 | 3 |
| Early withdrawal | 2 | 1 | 1 |

Overall response among these patients was PR in 2, tumor regression in 2, mixed response in 1, stable disease greater than 4 months was seen in 5. Six patients had progressive disease. Among the responding patients, one had biopsy with no evidence of viable tumor (FIG. 1). One patient decided to stop therapy after 10 months and remained disease free for another 16 months. FIG. 2 depicts scans for a patient with laryngeal SCC treated weekly with 10 mg/kg sEphB4-HSA. Scans show partial response at week 8 of therapy.

Examples of patients showing tumor response or disease control are depicted in FIG. 1, FIG. 2, and FIG. 3 and Tables 2-4.

TABLE 2
Patient: EH 07 76F Tumor: Tonsillar SCC.
Well differentiated. Keratinizing.

| | Treatment | Best Overall |
|---|---|---|
| Regimen Number | (History) | Response |
| 1 | RT + Chemo. Cisplatin | CR |
| | or Taxol + carbo | |
| 2 | Tarceva | PR |

TABLE 3
Patient: AC 69M Tumor: Laryngeal SCC.

| Regimen | Treatment | Best Overall |
|---|---|---|
| Number | (History) | Response |
| 1 | Radiation/chemo | Relapsed in nasopharynx |
| | | after 11 months |
| 2 | Surgery, carboplatin, | PD in 4 months |
| | 5FU, Erbitux | |
| 3 | Paclitaxel | PD in 7 months |

TABLE 4
Patient: RP 61M Tumor: Tonsillar SCC (HPV−) Poorly
differentiated. Non keratinizing. sEphB4-HSA
(5 mg/kg weekly - 4 cycles/4months).

| Regimen | Treatment | Best Overall |
|---|---|---|
| Number | (History) | Response |
| 1 | RT + Cisplatin + | PR, 2 months |
| | taxotere + 5FU | |
| 2 | Erbitux | Near CR, 5 months |
| 3 | Surgery- Bilateral | CR |
| | neck dissection | |
| 4 | Erbitux | 4 months, metastasis to |
| | | abdominal nodes and lung |
| 5 | Carboplatin + | Initial PR, PD in 5 months |
| | Taxol | |

This study demonstrates that sEphB4-HSA as single agent has activity in relapsed refractory HNSCC and suggests that sEphB4-HSA may be used as a first-line therapy for the treatment of HNSCC.

### Example 2: sEphB4-HSA Monotherapy in Hepatocellular Carcinoma

We have studied a cohort of HCC (18 years) with histologically confirmed advanced hepatocellular carcinoma. Patients previously treated with sorafenib and/or PD1 antibody were eligible. Primary endpoints were safety and tolerability and objective response rate (Response Evaluation Criteria in Solid Tumors version 1.1), duration of response, duration of stable disease and time to progression. Patients received intravenous sEphB4-HSA 10 mg/kg every week. 15 eligible patients have been accrued to the study. Most of the patients were Asian men and most had ECOG performance status of 1. All patients have previously been treated with systemic therapy, PD1 antibody in 10, Nexavar in 9, surgery in 6, and radiation in 5. Most patients had 2 or more prior regimens. The objective response rate thus far is 1 of 15 patients (7%), and stable disease greater than 4 months in 8 (greater than 20+months in 2). In conclusion, sEphB4-HSA can be given safely for long periods of time. Grade 3 toxicity include fatigue in 1, nausea in 1, neutropenia in 1, and hypertension in 6. Hypertension required dose reduction in 5. Durable objective response and long stable disease after Nexavar and PD-1 failure supports development as a single agent and in combination with PD-1 antibody.

TABLE 5
HCC Response Overview.

| RESPONSE | (N = 15) |
|---|---|
| Objective Response | |
| PR/CR | 1 |
| Stable disease | 8 |
| (Tumor regression) | (4) |
| Progressive disease | 6 |
| Disease control with | 8 |
| stable disease | |
| >6 mo. | 4 |

TABLE 6
HCC Response Patient Duration.

| Patient | Response | Duration | |
|---|---|---|---|
| FN | PR | 22+ | Mo |
| SH | SD | 4+ | Mo |
| TN | SD | 8+ | Mo |
| MO | SD | 6 | Mo |
| AP | SD | 2 | Mo |
| RT | SD | 4 | Mo |
| EK | PD. Fibrolamellar | 2 | Mo |
| HT | SD | 2+ | Mo |

In all, 8 patients had stable disease lasting 4 or more months and 2 patients over 20 months. Grade 3/4 treatment-related adverse events were seen in 7 patients (47%) patients, 6 of which were hypertension. Two patients required dose reduction. No patient had any complications from hypertension.

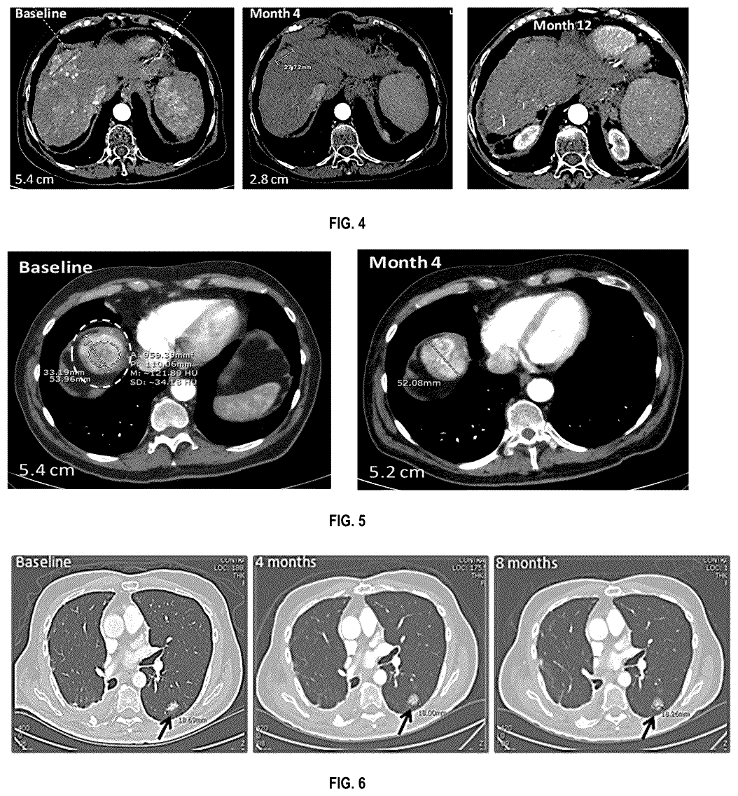

One patient (FN), a 79-year old female with HCV related HCC, was previously treated with TACE for 15 months which provided a partial response. She was then treated with PD1 antibody and progressed after 5 months. She was then treated with sEphB4-HSA for 11 months and had two small residual nodules treated with stereotactic radiation therapy. The patient currently remains disease free off therapy for 8+months (or 22+months from initial study entry) (FIG. 4).

TABLE 8
Patient: FN 79F Tumor: HCC

| Regimen | Treatment | Treatment | | Best Overall |
|---|---|---|---|---|
| Number | (History) | Duration | | Response |
| 1 | TACE | 15 | months | PR |
| 2 | Anti-PD1 Antibody | 5 | months | SD |
| | Therapy (Nivolumab) | | | |
| 3 | sEphB4-HSA | 11 | months | PR |
| 4 | MWA/SBRT | 1 | month | TBD |

Another patient (BW), a male with HCV related HCC, was previously treated with TACE with stable disease for 7 months. He then received PD1 antibody with tumor regression. He subsequently had disease progression including lung metastasis. He remained on PD1 antibody for 18 months duration while slowly progressing after the first 8 months. He has been on sEphB4-HSA for 20+months with stable disease. A third patient (TN) with HCV had liver transplant and recurrent HCC. He received Nexavar, TACE, Gemzar, Oxaliplatin, and Yttrium-90 in the past. He had stable disease for 8 months (FIG. 5).

TABLE 9
Patient: BW 66M Tumor: HCC

| Regimen | Treatment | Treatment | Best Overall |
|---|---|---|---|
| Number | (History) | Duration | Response |
| 1 | TACE with Nexamar | 7 months | SD |
| 2 | Anti-PD1 Antibody | 18 months | 13% |
| | Therapy (Nivolumab) | | reduction |
| 3 | MWA & yttrium-90 | 3 months | PD |

This phase 2 study demonstrates that treatment with sEphB4-HSA resulted in tumor regression and durable disease control after failure to Nexavar and PD1 antibody and provide improved response, duration of response and survival and suggests that sEphB4-HSA may be used as a first-line therapy for the treatment of HCC.

### Example 3: sEphB4-HSA Monotherapy in Kras mutant Non-small Cell Lunq Adenocarcinoma

A cohort of Kras mutant adenocarcinoma lung patients were evaluated in a single agent sEphB4-HSA trial. Patients were included if they had prior diagnosis of KRAS mutant lung cancer, had failed prior therapies and had evidence of progressive disease. 9 patients have been accrued. 2 of the 9 patients came off the study within 4 weeks of therapy and thus were not evaluated for response. Summary of the patients is included in the clinical summary data as well. Each of the five cases are summarized below:

Patient AC: Kras mutant multifocal adenocarcinoma lung and brain metastasis. Patient was treated with cranial irradiation, and subsequently with carboplatin, paclitaxel, Avastin for 3 cycles. Patient was intolerant and progressed in 7 months. He was treated with sEphB4-HSA and had stable disease for 11 months (FIG. 6).

TABLE 10
Patient AC treatment overview.

| | Avastin; Paclitaxel; | sEphB4- |
|---|---|---|
| Treatment | Carboplatin | HSA |
| Outcome | SD 7 months | SD 11 months |

Patient TC: Kras mutant Right upper lung adenocarcinoma, received Alimta and carboplatin, progressed after 10 months. He then received nanosphere docetaxel for 6 months with stable disease, Taxotere for 4 months with progressive disease. He went on sEphB4-HSA, had resolution of right shoulder pain, and stable disease for 8 months.

TABLE 11
Patient TC treatment overview.

| | Alimta; | Nano- | | sEphB4- |
|---|---|---|---|---|
| Treatment | Carboplatin | taxotere | Taxotere | HSA |
| Outcome | 10 mo | 6 mo | 4 mo | 8 mo |

Patient PS: Kras mutant lung cancer treated with Alimta carboplatin and avastin for 6 cycles, followed by alimta maintenance for a total of 21 months. At relapse he was treated with taxotere for 8 months, avastin with progressive disease, intolerant to Novelbine and avastin, progressive disease on avastin alone, progressive disease on etoposide plus cisplatin gemcitabine. He then received gemcitabine plus avastin for 6 months. He went of sEphB4-HSA trial with progressive disease. He has stable disease for 4 months.

TABLE 12
Patient PS treatment overview.

| | Avastin; | | | | | | |
|---|---|---|---|---|---|---|---|
| | Alimta | | | Novelbine | Etoposide | Gemsar | sEphB4- |
| Treatment | Carboplatin | Taxotere | Avastin | avastin | Cisplatin | Avastin | HSA |
| Outcome | 21 mo | 6 mo | PD | Intolerance | PD | PD | 4 mo |

Patient JC: 76-year old woman with Kras mutant moderately differentiated adenocarcinoma right upper lung, had surgery, and adjuvant cisplatin and Alimta for 4 cycles. Patient had disease progression. Tumor was PD-L1 positive (70%), thus went on Pembrolizumab trial. Tumor progressed after 3 months. She was put on sEphB4-HSA trial and had stable for 4 months.

TABLE 13
Patient JC treatment overview.

| | | | | |
|---|---|---|---|---|
| | | Adjuvant alimta; | | sEphB4- |
| Treatment | Surgery | Cisplatin | Pembrolizumab | HSA |

| | | | |
|---|---|---|---|
| Outcome | 3 mo | 3 mo | 4 mo |

Patient HW: Kras mutant lung cancer patient received Avastin, Alimta and Carboplatin. Patient progressed after 6 months and was placed on sEphB4-HSA trial. Patient had table disease for 32 weeks.

TABLE 14
Patient HW treatment overview.

| | Avastin; Alimta; | sEphB4- |
|---|---|---|
| Treatment | Carboplatin | HSA |
| Outcome | 6 mo | 8 mo |

7 of 9 evaluable patients for response, 5 patients showed stable disease on EphB4-HSA monotherapy for 11 months, 8 months, 8 months, 4 months, and 4 months, respectively.

### Example 4: sEphB4-HSA Monotherapy in Kaposi's Sarcoma

Three patients with KS were studied. Two had HIV and one was HIV negative. All three patients had previously been treated with multiple prior therapies. One of the two HIV KS patients who had six prior therapies had advanced disease over the legs and extensive long-standing associated edema that had not resolved completely on three prior therapies.

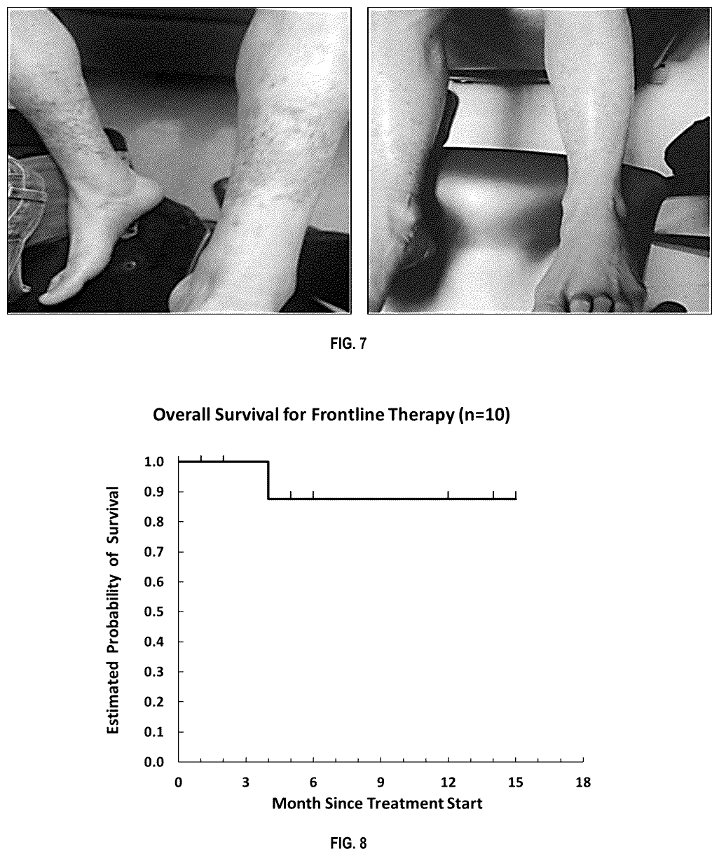

Another patient had previously been treated with cytotoxic chemotherapy and multiple investigations agents. He was put on sEphB4-HSA. He had complete resolution of the tumor and complete resolution of leg edema (FIG. 7). He continues to be in remission for over 2 years. Treatment frequency has been reduced to 10 mg/kg Q2 weeks with sustained remission for over 6 months. Based on preclinical, target expression in the tumor and response in the clinic, a phase II trial is now in progress through NCI-CTEP-AMC (AIDS Malignancy Consortium).

TABLE 15
Treatment regimen vs best response overview.

| | | Best |
|---|---|---|
| Regimen | Treatment | Response |
| 1 | IM862 | PD |
| 2 | Intralesional | PR |
| | vinblastine | |
| 3 | DaunoXome | PR |
| 4 | Taxotere | PR |
| 5 | Veglin | PR |
| 6 | Taxol | PR |

### Example 5: sEphB4-HSA Bladder Cancer First line or Frontline therapy

Advanced Disease: Newly diagnosed locally advanced (beyond the bladder or draining system, ureter, renal pelvis) bladder and urothelial carcinoma prior to use of systemic therapy or within 12 months of neo-adjuvant systemic chemotherapy (FIG. 8). Eight patients were ineligible to receive standard Cisplatinum containing regimen, and thus had very poor survival. Ten patients had been treated with sEphB4-albumin containing regimen at 10 mg/kg weekly without cytotoxic chemotherapy such as cisplatinum, carboplatin, gemcitabine, methotrexate. Six patients had completed the first six weeks of therapy and undergone tumor evaluation with radiological methods (computerized tomography) one or more times. Each of the six patients had a response defined by RECIST response criteria (version 1.0). Furthermore, each of the six patients achieved complete remission. Furthermore no patient had relapsed after a follow up of 4 to 16 months. Two patients who came off therapy within the first 3 weeks due to unrelated reasons died. Eight patients are alive.

Tumors with mutations in TP53, ARID-1, BAP-1, RAS, PBRM1, P13K, PIK3CA, did not prevent response to therapy.

Standard-of-care chemotherapy contains cisplatinum generally combined with gemcitabine. The best treatment regimen provided an overall response in around 40% of cases, progression free survival of 6-7 months, and overall median survival of approximately 16 months. Patients unable to receive cisplatinum (generally 40-60%) had much worse prognosis.

### Example 6: Muscle Invasive Bladder Cancer

FIG. 8 depicts a graphical representation of the overall survival of advanced bladder cancer, newly diagnosed and treated with sEphB4-albumin fusion protein containing regimen.

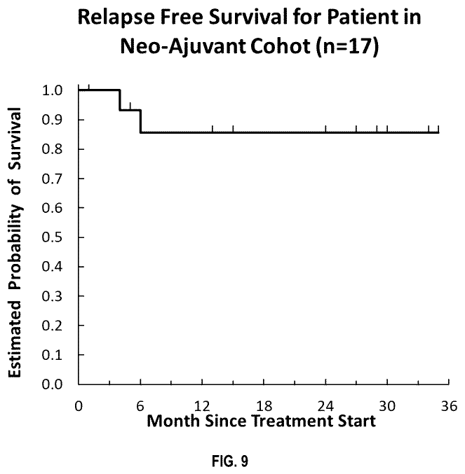

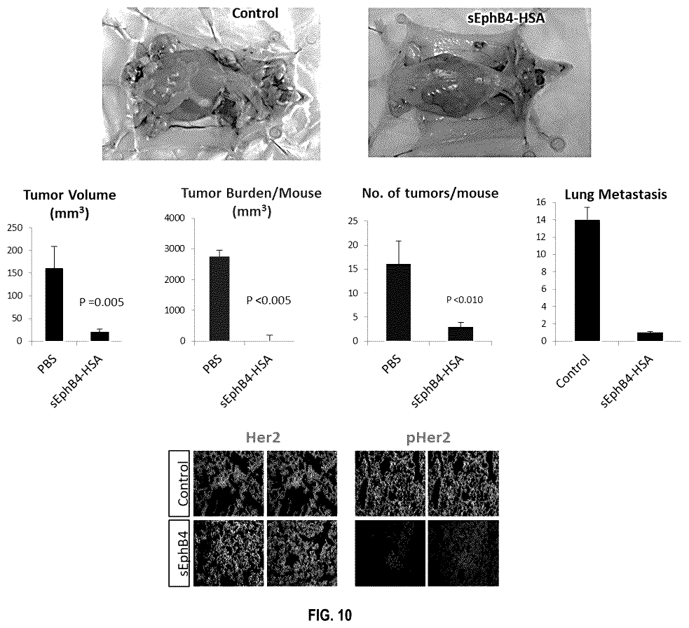

Newly diagnosed patients with bladder cancer received standard-of-care cisplatinum-gemcitabine chemotherapy, which provided pathologic complete remission at the time of curative surgery (radical cystectomy) of near 30%, predictive of long term disease free survival. Median time to relapse was around 14-17 months. We treated 17 patients with newly diagnosed muscle invasive bladder cancer, and treated with sEphB4-albumin fusion protein (FIG. 9). Among the 10 patients who expressed EphrinB2, the drug target, had a pathologic complete remission of 70%. None of these 7 patients had recurred with the longest follow up of 36 months. Furthermore, two patients who declined cystectomy remained free of disease after over 2 years of follow up, indicating that organ sparing can be achieved in biomarker positive bladder cancer patients. These results were highly unexpected. Furthermore the 7 biomarker EphrinB2 negative patients, none of which had relapsed after following up of up to 36 months, while only 2 patients had achieved pathologic complete remission. These data suggested that the EphrinB2 may have been induced during therapy leading to biological benefit, even generation of memory response.

### Example 7: Muscle Non-Invasive Bladder Cancer or Superficial Cancer

Biomarker positive patient tumors, such as muscle invasive bladder cancer, are even more likely to have gained durable responses having a tumor that responded to immune therapy more than muscle invasive and metastatic bladder cancer. Notably BCG is highly active in muscle non-invasive bladder cancer. HER2/EGFR2 mutant cancers of various organs: LUNG, HEAD and NECK, BLADDER, and Her2 mutant tumors are not curable. Standard therapy includes chemotherapy, kinase inhibitors, and Her2 antibody drug conjugates. These therapies produced partial responses in a partial patient population.

Her2 localizes in the cell membrane with EphB4. Her2 induces EphB4. Her2-directed antibody reduced EphB4 levels. Work showed that EphB4 regulated Her2 downstream signaling and phosphorylation. To begin, we investigated Her2 transgenic mice which, as expected, had high EphB4 expression. Treatment of transgenic mice with soluble EphB4 blocked tumor formation and metastasis including lung (FIG. 10). Mice were treated for five weeks at a dose of 7.5 mg/kg three times weekly via IP injection. Tumor tissues were analyzed for Her2/ERBB2 total protein and protein phosphorylation. Tissue analysis showed reduction of Her2 phosphorylation via fluorescence confocal microscopy staining analysis. We thus treated Her2 over-expressing tumors after failure to chemotherapy. Rapid and often complete remission was observed with long durability. sEphB4-HSA treatment in mice showed a statistically significant (p=0.005) reduction in tumor volume, as well as a significant (p<0.005) reduction in tumor burden per mouse. Following sEphB4-HSA treatment, a statistically significant decrease in the average number of tumors per mouse (p<0.01) was observed. Additionally, the amount of lung metastasis observed were greatly diminished.

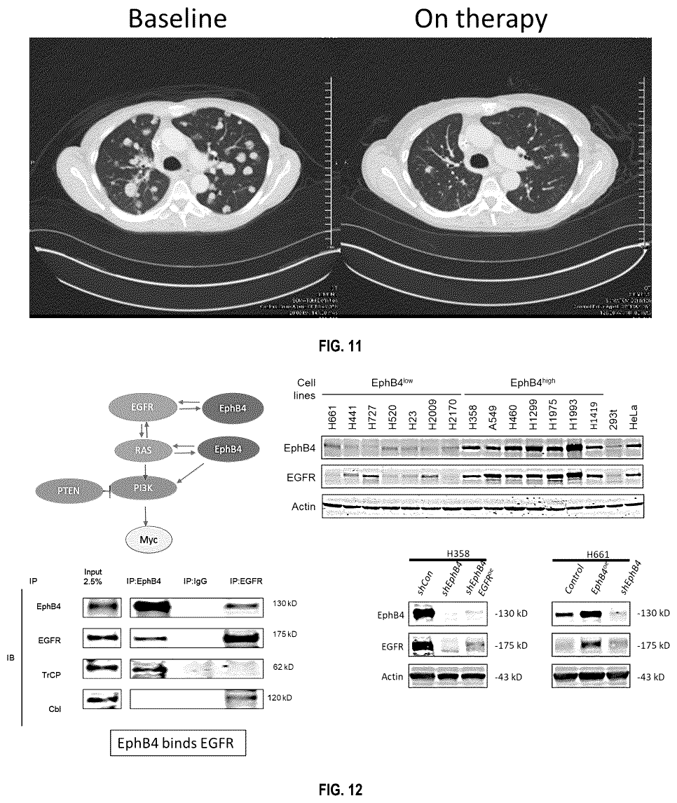

In Humans Her2 mutant tumors provide an even greater challenge. We treated patients with Her2 mutant cancers after failure to standard-of-care chemotherapy and kinase inhibitors. This state of disease represents the unmet need. We treated five patients with Her2 mutation including exon 20 p 772_A775 duplication which poses resistance to therapy, concurrent mutations in RB1 Exon20 pL700X, TP53 Exon 4 p.S116fs; ERBB2 Exon 17 V659E, concurrent PIK3CA E 545K, TP53, exon 5, R158fs, ATM G2891D NF1 E2143; in another case, the ERBB2 mutation has concurrent ATM, RICTOR, CCNE1, CDKN18, IRS2, PMS2, TERT, TP53. The patient had complete remission and remained disease free after 2 years and off therapy. FIG. 11 illustrated the ERBB2 exon 20 duplication response to sEphB4 therapy.

In another patient, in addition to ERBB2 mutation, the patient had concurrent mutation in ALK and ROS1 rearrangement. Tumors were localized to the lung, head and neck, bone, brain, and lymph nodes.

In each patient, we observed rapid response with sEphB4-albumin fusion protein given at 10 mg/kg weekly therapy. Complete remission in three of five patients was observed. Four of five patients had response, Three patients achieved complete remission, with durability of 6 months to over 2 years and ongoing.

### Example 8: EGFR Mutation and High Expression Pose Major Clinical Challenge

Increased EGFR is observed in many cancers including head and neck cancer, lung cancer, colon cancer, bladder cancer, and many others. Response to single agent antibody therapy is relatively low and lasts for short duration. EGFR gene mutation is an even bigger challenge and failure to kinase inhibitor is generally the rule. There is need for additional therapies especially early in therapy and combined with EGFR targeted therapy.

We determined that EGFR and EphB4 enhance each other's expression. As FIG. 12 illustrates, immunoprecipitation (IP) with anti-EphB4 pulled down EGFR, and likewise, IP with anti-EGFR pulled down EphB4, demonstrating that the two co-localized via direct binding. EphB4 knock-down lowered EGFR protein levels, as demonstrated by immunoblotting of cell lysates. from. Targeting each agent showed efficacy in EphB4 overexpressed NSCLC cells (H358 non-small cell lung cancer (NSCLC) cell line, which harbors a KRAS mutation), and the activity is enhanced when combined even in H661 (Her2 overexpressed) NSCLC cells. Thus sEphB4 and EGFR targeted therapies showed potent synergistic activity, forming the basis for combined use.

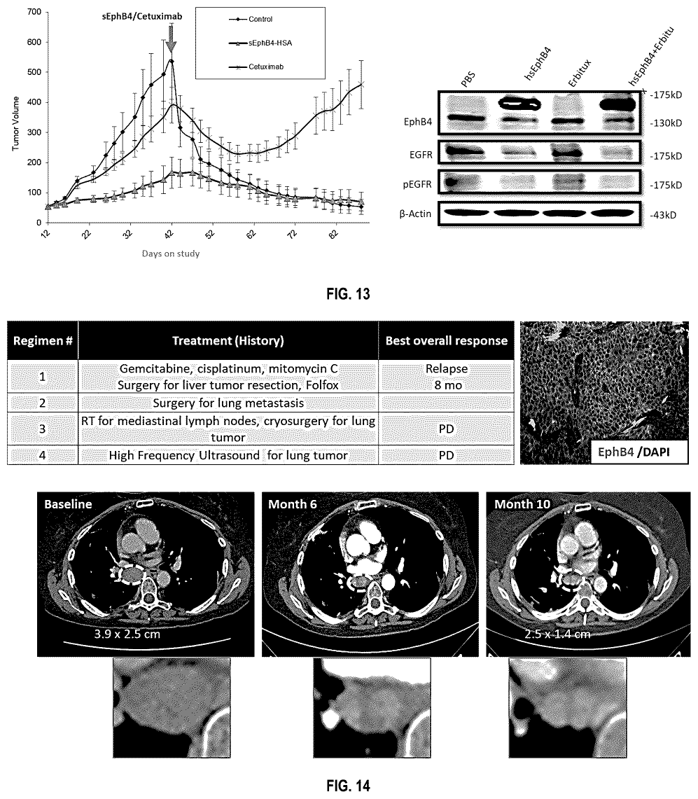

As demonstrated in FIG. 13, in vivo efficacy studies with sEphB4 and anti-EGFR Ab (cetuximab) showed synergistic efficacy. In tumors resistance to anti-EGFR treatment, sEphB4 was effective, with sEphB4+cetuximab more effective than either treatment individually.

### Example 9: Cholangiocarcinoma Outcomes from sEphB4-HSA treatment

Cholangiocarcinoma has poor response to therapy. Standard chemotherapy is cisplatinum and gemcitabine. There is need for novel therapies, especially targeted therapies. Recently, FGFR-mutant cholangiocarcinoma treatment with kinase inhibitor has shown tumor regression, however the response rates are low and complete remissions are even less likely. There is need for novel therapies. We have treated cholangiocarcinoma with sEphB4 and select patients showed durable response. This represented a solution for an unmet need.

As shown in FIG. 14, provided are examples of responses in cholangiocarcinoma, for example, a 64 year old female who had been previously treated with gemcitabine, cisplatinum, mitomycin C, surgery, radiation therapy, and high frequency ultrasound had progressed with lung metastasis. She was treated with sEphB4-albumin fusion protein given at 15 mg/kg dose every two weeks and had a substantial tumor regression which continued for over one year.

TABLE 16
Treatment overview and response for 64-
year old female with cholangiocarcinoma.

| | Treatment | Best overall |
|---|---|---|
| Regimen # | (History) | response |
| 1 | Gemcitabine, cisplatinum, | Relapse |
| | mitomycin C Surgery for liver | 8 mo |
| | tumor resection, Folfox | |
| 2 | Surgery for lung metastasis | |
| 3 | RT for mediastinal lymph nodes, | PD |
| | cryosurgery for lung tumor | |
| 4 | High Frequency Ultrasound | PD |
| | for lung tumor | |

### Example 10: EphB4 Expression Confers a Growth Advantage to Kras Mutated Cells

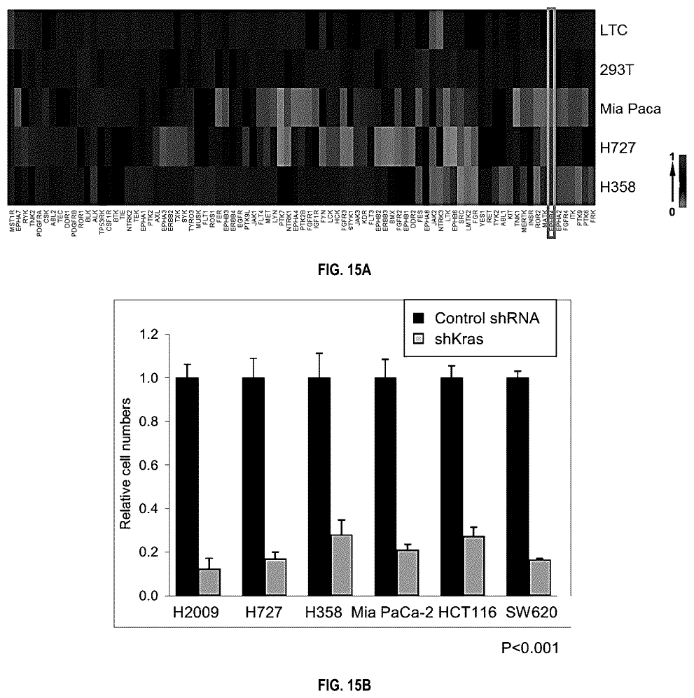

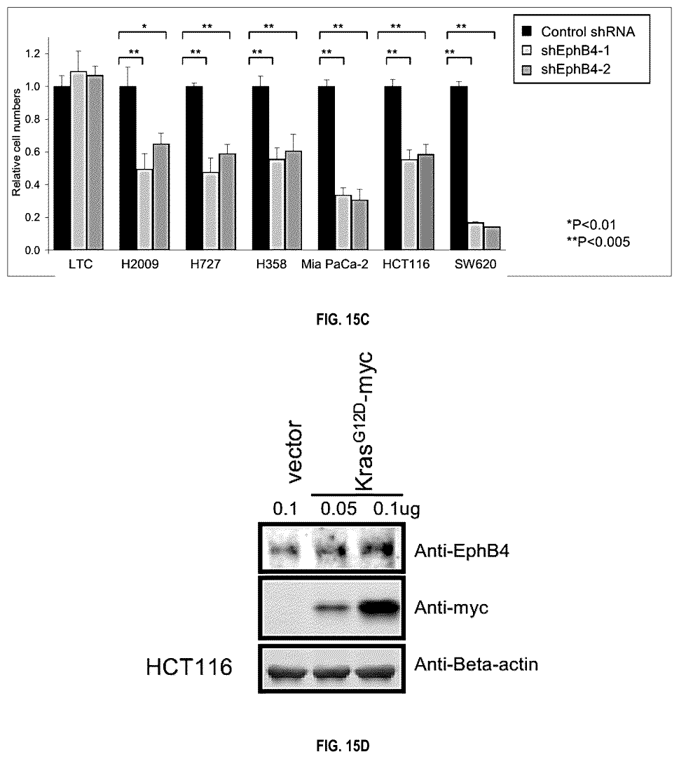

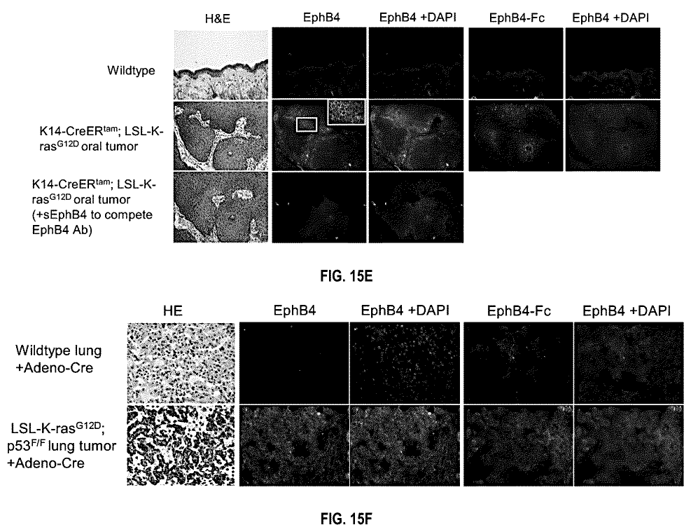

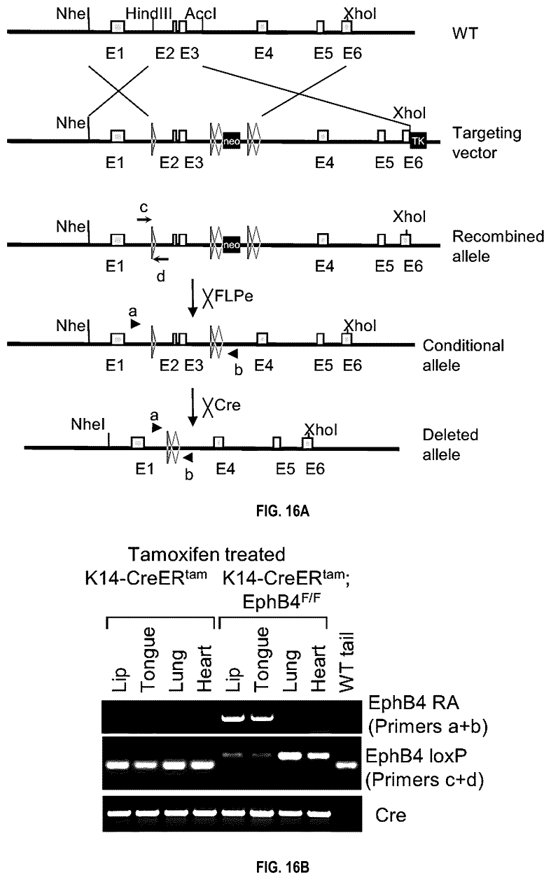

We used the human tyrosine kinase siRNA library (Thermo Scientific) to investigate the role of tyrosine kinases in modulating Kras-mutated cancer cells viability. Three Kras-mutated cancer cell lines (H358, H727, and Mia Paca-2) and two Kras wild-type cell lines (293T and LTC) were transfected with SMARTpool siRNA library (a mixture of 4 siRNAs/gene) targeting 85 tyrosine kinases. The MTT assay results were presented as a heat map indicating the effect of siRNA-mediated knockdown of tyrosine kinases on the survival of cell lines (FIG. 15A). Receptor tyrosine kinase EphB4 is the target of interest because inhibition of EphB4 resulted in one of the most reduced viability in three Kras-mutated cells with the best p-value compared to controls (293T and LTC) (P=0.018) (FIG. 15A).

We confirmed that EphB4 is a key regulator of cell survival in Kras-mutated and dependent cancer cell lines. We analyzed the following cell lines, which harbor oncogenic Kras mutations as shown in Table 17, for their Kras dependency: non-small cell lung cancer cell lines (NSCLCs) (H358, H727 and H2009), pancreatic carcinoma cell line (Mia Paca-2), and colon cancer cell lines (HCT116 and SW620). All six cell lines were sensitive to Kras depletion (FIG. 15B). We furthermore knocked down EphB4 in the cells by two shRNAs targeting different regions of EphB4. The results showed that EphB4 was required for the viability of Kras-mutated cell lines, whether or not in the presence of TP53 mutations (FIG. 15C and Table 17).

TABLE 17
Cell line mutations and cancer type overview.

| Cell line | Cancer Type | Mutations |
|---|---|---|
| LTC | KSHV-infected | Kras WT; TP53 WT |
| | endothelium | |
| H358 | Lung | Kras^{G12C} |
| H727 | Lung | Kras^{G12V}; TP53^{Q165, S166insYKQ} |
| H2009 | Lung | Kras^{G12A}; TP53^{R273L} |
| Mia Paca-2 | Pancreas | Kras^{G12C}; TP53^{R248W} |
| HCT116 | Colon | Kras^{G13D} |
| SW620 | Colon | Kras^{G12V}; TP53^{R273, P309S} |

The expression of EphB4 protein is induced in a variety of human cancers and is associated with advanced tumor stages. We overexpressed KrasG12D-myc in HCT116 cells and showed that the level of endogenous EphB4 protein was enhanced in a dose-dependent manner by Kras (FIG. 15D). We also examined EphB4 expression in tumors of two different Kras-driven mouse cancer models (oral papillomas and NSCLC). The K14-CreERtam;LSL-KrasG12D mice express a tamoxifen inducible Cre recombinase (CreERtam) driven by cytokeratin 14 (K14) promoter. They also harbor a mutated Kras (LSL-KrasG12D) and develop oral papillomas one month after tamoxifen induction (Cre-mediated removal of the loxP-flanked STOP cassette (LSL) upstream of mutated KrasG12D). Immunostaining showed that EphB4 and its ligand ephrinB2 were both increased in the tumors (FIG. 15E). Elevated EphB4 staining was observed in basal and intermediate layers of papillomas and ephrinB2 ligands were expressed complementary to EphB4 receptor in the more differentiated tumor area.

In the NSCLC model, we delivered Cre recombinase by using adenovirus (Adeno-Cre) to the lung cells of LSL-KrasG12D;P53F/F mice. The mice develop lung adenocarcinoma after adenovirus intratracheal infection. Overexpression of EphB4 and ephrinB2 were also observed in the tumors (FIG. 15F). These results suggested that EphB4 signaling is induced by oncogenic KrasG12D.

### Example 11: Genetic Ablation of EphB4 Increases Survival in Kras Mutant Mice

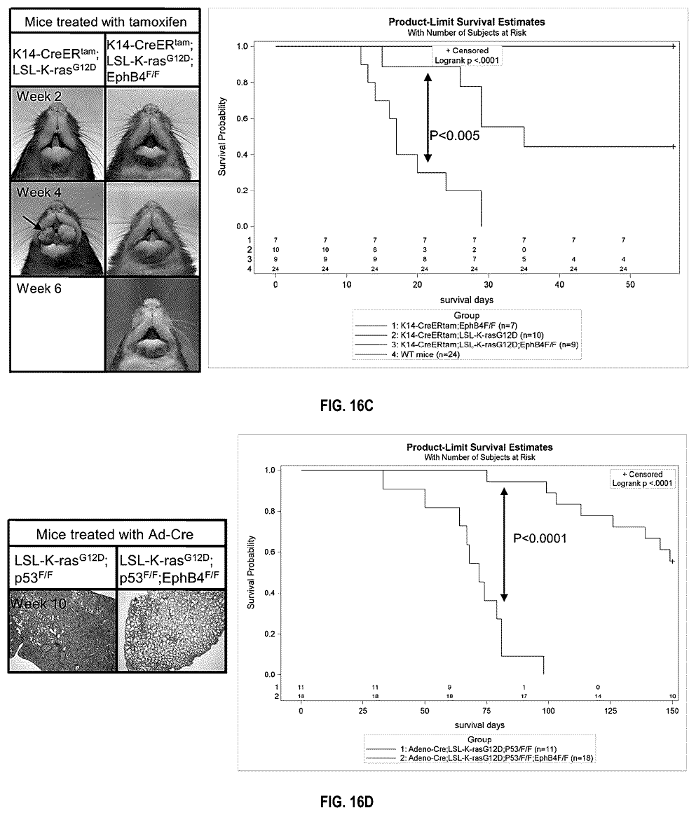

To study the role of EphB4 in tumor development, we generated EphB4 conditional knockout mice targeting exon 2 and 3 of ephB4 gene. The mutant creates a premature stop codon in ephB4 after cre-mediated recombination (FIG. 16A). To determine the tissue-specific knockout of EphB4F/F, we crossed the mutants with K14-CreERtam mice and treated mice with tamoxifen. The DNA samples from lip, tongue, lung, and heart were collected and genotyped one month after tamoxifen treatment. EphB4 rearrangement (EphB4 RA in FIG. 16B) was detected only in the lip and tongue of K14-CreERtam;EphB4F/F mice, as expected. The removal of STOP cassette upstream of Kras gene in K14-CreERtam;LSL-KrasG12D mice were also checked in the lip tumor.

The conventional EphB4 knockout mice reported previously exhibit embryonic lethality at E10 due to cardiac defects. We therefore bred conditional EphB4 mutants to ubiquitously expressed CMV-Cre deletion mice which produced a complete gene knockout. Growth retardation of CMV-Cre;EphB4F/F embryos was noted at E10.5 and E11.5 stages. We also crossed EphB4F/F with tamoxifen inducible CMV-Cretam mice to check the significance of EphB4 in adults. Pathology analyses of major organs of CMV-Cretam;EphB4F/F mice, including lung, heart, kidney, liver, and small intestine, were performed one month after tamoxifen induction. The induced mice appeared healthy and viable with no noticeable phenotype observed in the organs of mutants compared to that of control. These results suggested that, although EphB4 is critical for embryo development, it had no significant function in normal adults.

In addition to the oncogenic properties of the overexpressed EphB4 in many human cancers, knockdown of EphB4 reduced cell viability of Kras-dependent cell lines. These results prompted investigation into if EphB4 affected tumorigenesis in Kras-driven cancer models. To this end, K14-CreERtam;LSL-K-rasG12D;EphB4F/F (K14KB4) mice were generated and compared to K14-CreERtam;LSL-K-rasG12D (K14K) mice. Oral squamous papillomas were detected in 100% of K14K mice (n=10) in the 4th week after tamoxifen treatment. The results showed that K14KB4 (n=9) mice had significantly less tumor growth and extended survival (FIG. 16C) compared to K14K (P<0.005). We observed even larger differences in survival in the NSCLC mice model. LSL-KrasG12D;P53F/F or LSL-K-rasGl2D;p53F/F;EphB4F/F mice were administered with Ad-Cre (AdKP and AdKPB4, respectively). The carcinogenesis of lung adenocarcinoma was dramatically reduced in AdKPB4 (FIG. 16D). More than half (56%) of the AdKPB4 (n=18) survived to day 150 after Ad-Cre infection, while all of the AdKP (n=11) died before day 98 (P<0.0001).

### Example 12: Knockdown of EphB4 Attenuated AKT and ERK Signaling in Kras-Driven Tumors

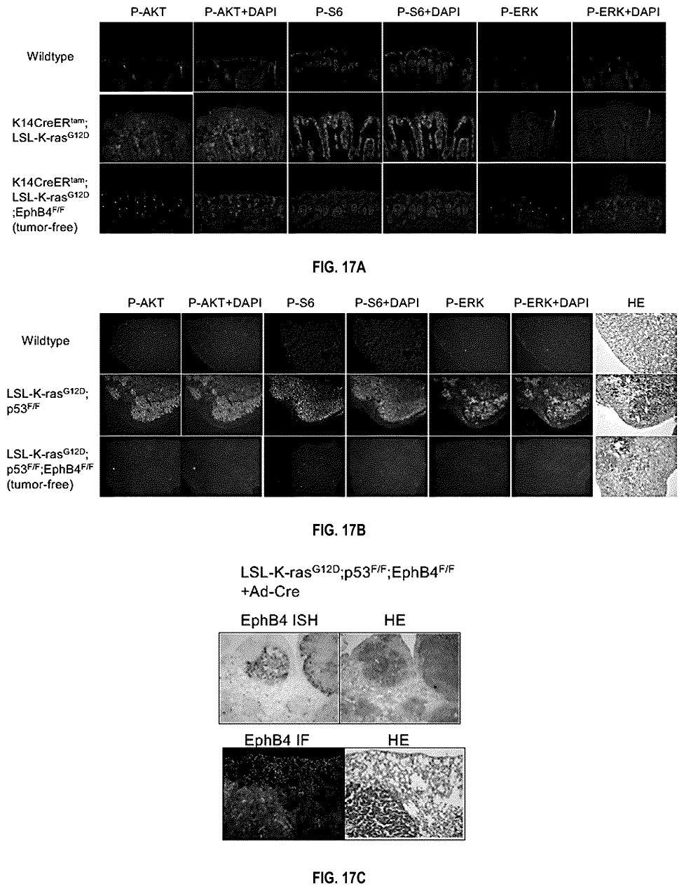

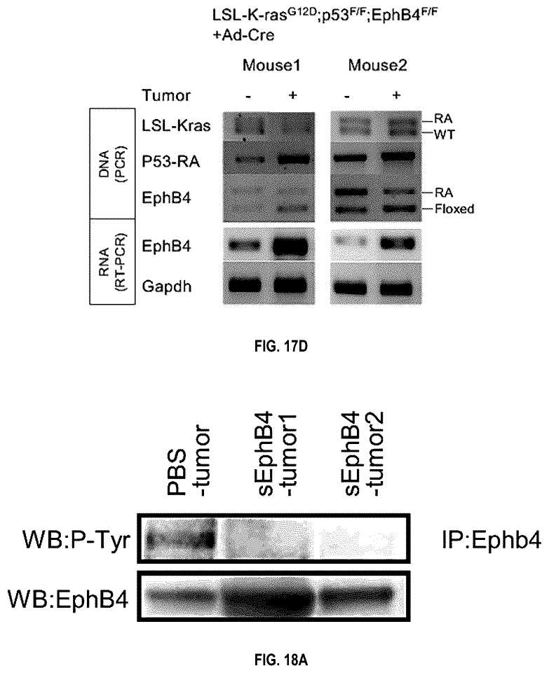

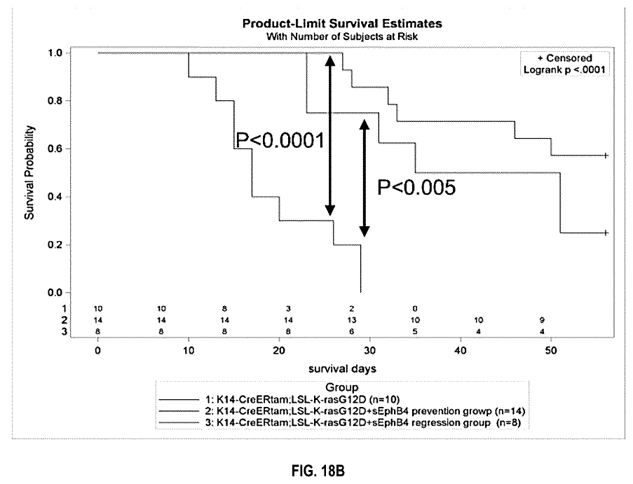

It is well established that oncogenic Kras activates P13K/AKT and MAPK/ERK signaling pathways, which both serve as important therapeutic targets in cancer treatment. We therefore investigated the expression levels of the p-AKT and p-S6 for activated P13K/AKT pathway and the levels of p-ERK1/2 for MAPK/ERK pathway in the mouse oral papilloma and lung adenocarcinoma. All signaling indicators, except for p-ERK1/2 which cannot be detected in oral papillomas, were significantly increased in tumor areas, but not in the tissues of EphB4 knockout mice (FIG. 17A for oral papilloma and FIG. 17B for lung adenocarcinoma).

We observed that, although EphB4F/F slowed tumorigenesis in both oral papilloma and lung adenocarcinoma mouse models, tumors at various levels eventually occurred in the K14KB4 or AdKPB4 mice. We proposed that it could be a result of incomplete knockout of EphB4 in the tumors. We first examined EphB4 expression in the lung tissues of AdKPB4 mice. Both in situ and immunofluorescence staining showed overexpressed EphB4 mRNA and protein, respectively (FIG. 17C). We also microdissected lung frozen sections from AdKPB4 and found that rearranged (RA)-Kras, RA-P53, and RA-EphB4 could be detected by PCR in both tumor and non-tumor areas, indicating Ad-Cre activity in the whole lungs (FIG. 17D, upper panel). However, the existence of the floxed EphB4 gene provided the evidence of incomplete knockout, because the band of floxed EphB4 would be totally lost if all the floxed alleles were deleted. Furthermore, RT-PCR clearly demonstrated overexpressed EphB4 mRNA in the tumor compared to non-tumor areas, suggesting EphB4 expression cannot be successfully decreased in certain areas of the lung, and therefore the tumorigenesis induced by mutated Kras and P53 cannot be blocked in AdKPB4 mice (FIG. 17D, lower panel).

We analyzed the RNA expression of EphB4 and EphrinB2 in the oral papilloma using in situ hybridization. The results showed that the RNA expression of EphB4 and EphrinB2 were much weaker but can still be detected in the tumor area of K14KB4 mice. Immunostaining revealed partial expression of EphB4 protein in the tumors of K14KB4.

### Example 13: Pharmacologic Inhibition of EphB4 Effectively Inhibits Kras-Driven Tumorigenesis In Vivo

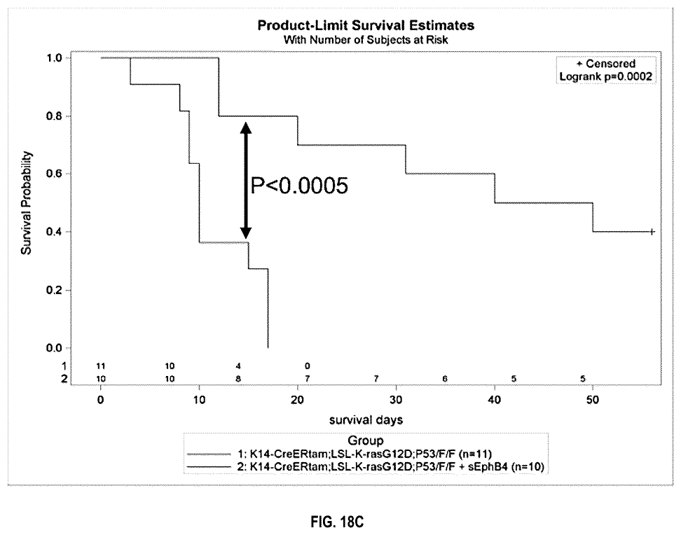

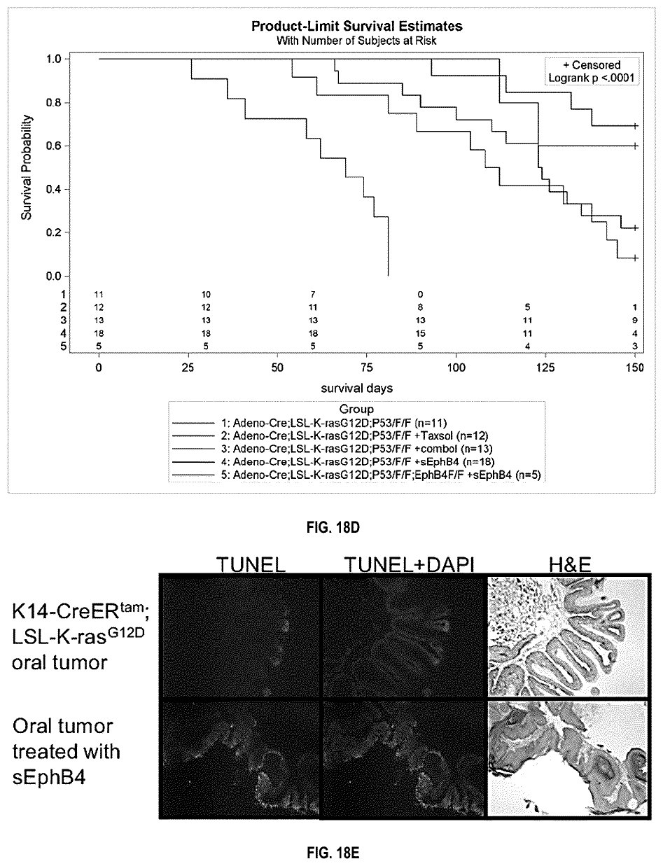

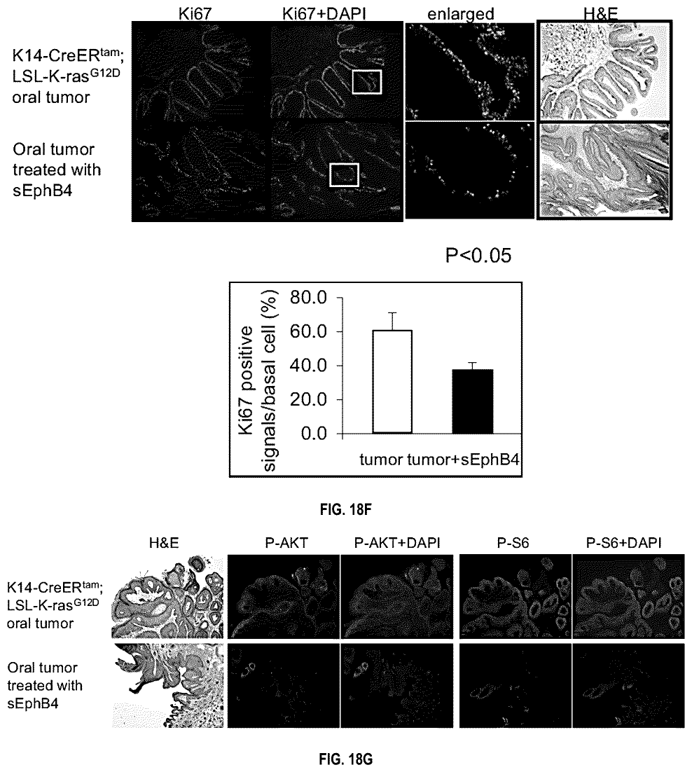

To further demonstrate the efficiency of sEphB4, we examined the tyrosine auto-phosphorylation status in activated EphB4 protein under sEphB4 treatment. We intraperitoneally injected sEphB4 (50 mg/Kg of mouse weight) to tamoxifen induced K14K mice. Three days after sEphB4 treatment, oral papilloma was collected, and the tumor lysates were immunoprecipitated with anti-EphB4 antibody. Western blotting showed that the p-Tyr signal of EphB4 was significantly decreased in the sEphB4 treated tumors, but not in the control (FIG. 18A). The results indicated that sEphB4 is able to block autophosphorylation, as well as the activation of EphB4 receptor in vivo.

We tested the therapeutic potency of sEphB4 by using the oral papilloma and NSCLC mouse models. K14K mice were treated with sEphB4 (20 mg/Kg, every other day) either starting at the same time with (prevention group) or two weeks after (regression group) tamoxifen induction. The survival rates of both sEphB4 treated groups were significantly increased compared to that of control K14K mice (FIG. 18B). Adding additional P53 knockout mutation to K14K mice (K14KP) enhanced tumor development. Prevention treatment of sEphB4 to K14KP also slowed tumorigenesis and prolonged survival (FIG. 18C).

The chemotherapy drug paclitaxel (Taxol) is widely used for the treatment of NSCLC. It has demonstrated synergistic interaction with other cancer drugs. We used our sEphB4 in combined with Taxol to treat the NSCLC mice model AdKP. Taxol and sEphB4 single drug treatments showed similar significant survival advantage compared to control, whereas no significant difference between Taxol-treated and sEphB4-treated mice was observed. The taxol and sEphB4 combination treatment further greatly improved survival in comparison with each single treatment (FIG. 18D).

Kras mutation has been suggested to be associated with decreased apoptosis and increased proliferation in tumors. To understand the effects of sEphB4 in the Kras-driven cancer, we examined the status of apoptosis and proliferation in the sEphB4 treated K14K mice harboring spontaneous tumors by using TUNEL assay and Ki67 staining, respectively. We found that sEphB4 treatment every other day for 20 days significantly increased apoptosis and decreased proliferation in tumors (FIG. 18E and FIG. 18F). Kras downstream signaling molecules p-AKT and P-S6 were also greatly reduced in the treatment of sEphB4 (FIG. 18G). Furthermore, short-term (44 hr.) tumor tissue culture confirmed the striking effect of sEphB4 on inducing tumor cell apoptosis.

### Example 14: EphB4 interrupts 8-TrCP1-mediated ubiquitination and degradation of Kras

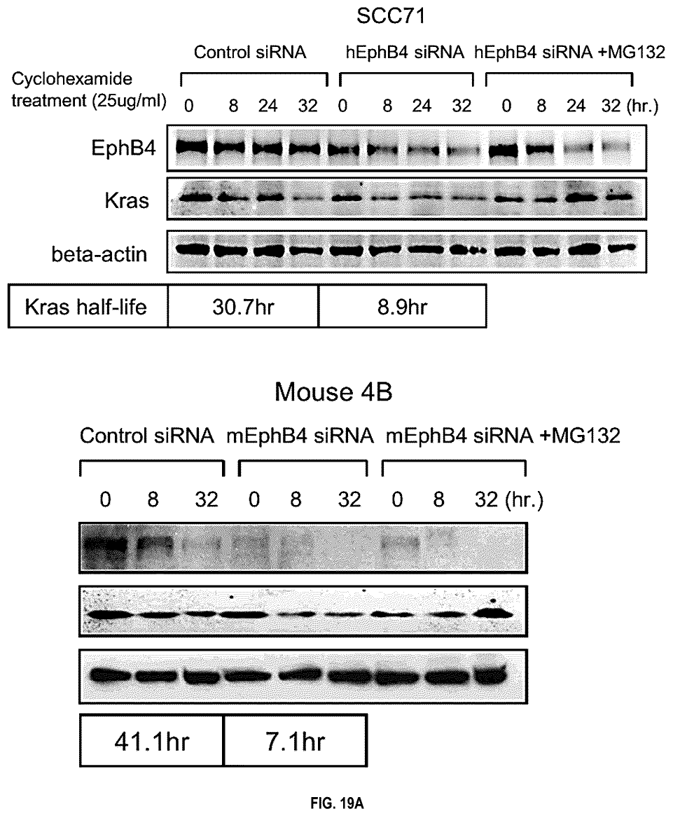

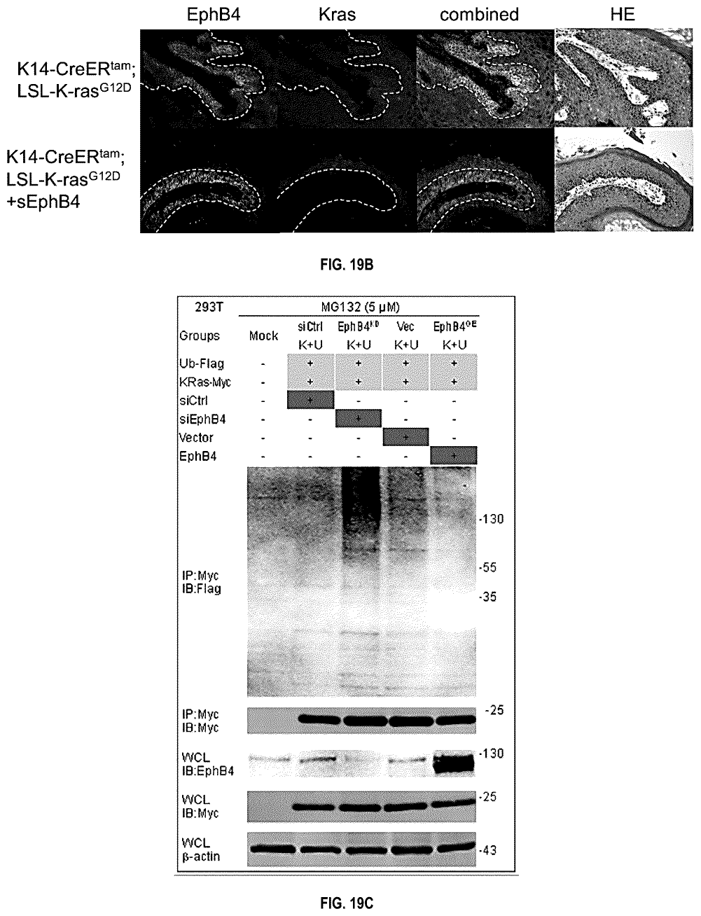

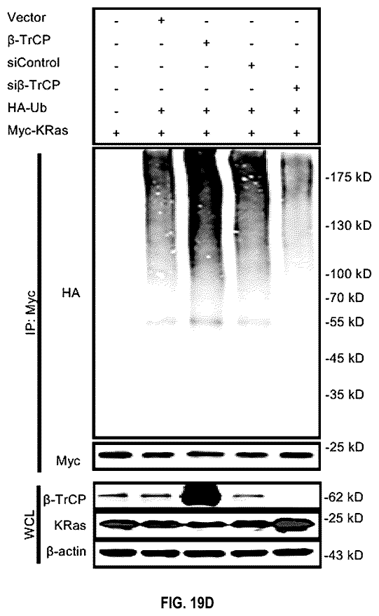

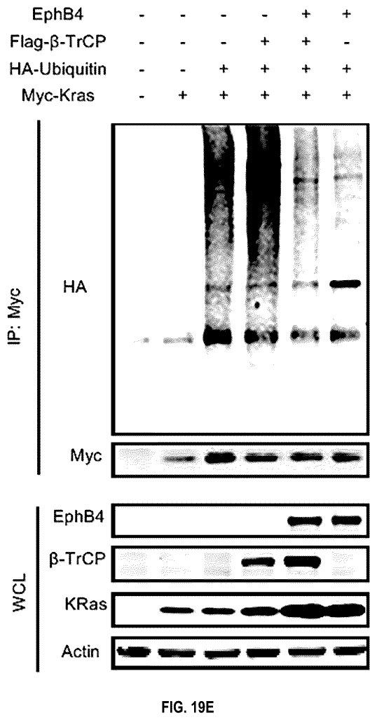

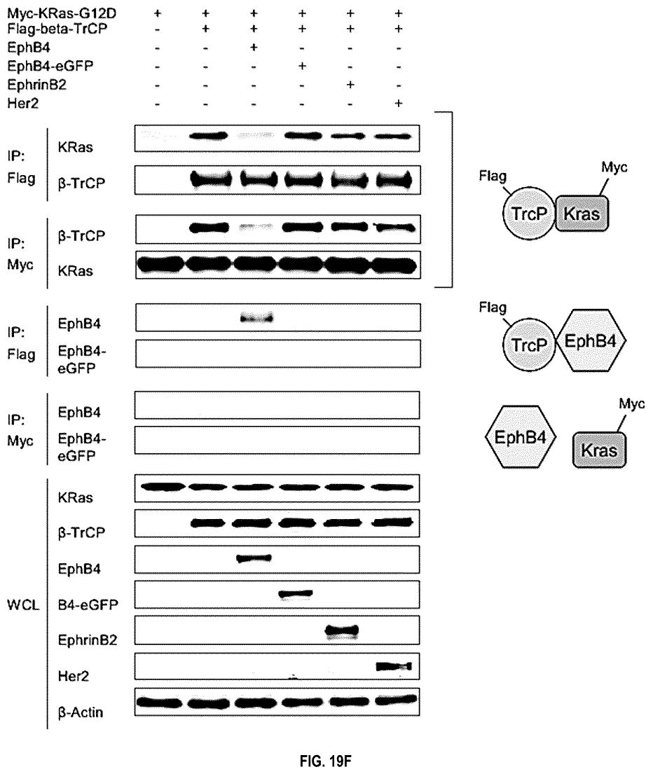

We have demonstrated that knockdown of EphB4 signaling either by mice genetic modification or by treating its antagonist, sEphB4, effectively eliminated Kras-driven tumorigenicity. Meanwhile, we found that knockdown of EphB4 by siRNA in HCT116 cell line decreased the levels of CMV promoter-driven overexpressed Ras proteins, which suggests that EphB4 may affect Ras protein stability. We determined whether EphB4 regulated the half-life of endogenous Kras protein. The human oral squamous carcinoma cell line, SCC71, which harbors wildtype Kras and the mouse NSCLC cell line, 4B-GFP, which harbors oncogenic KrasG12D were chosen to see if both wildtype and mutated form of Kras can be regulated by EphB4. We found that knockdown of EphB4 by siRNA decreased endogenous Kras protein half-life from 30.7 to 8.9 hrs. in SCC71 and from 41.1 to 7.1 hrs. in 4B-GFP cells (FIG. 19A). Moreover, the reduction of Kras half-life can be rescued by proteasome inhibitor MG132, suggesting that ubiquitin-proteasome machinery plays a major role in the control of Kras protein degradation. We further checked the Kras level in the tumor of K14K mice and found that, after sEphB4 treatment, Kras expression was decreased in the basal and intermediate layers of papillomas that correspond to the EphB4 overexpression cells (FIG. 19B).

The results of Kras half-life study prompt us to hypothesize, without wishing to be bound by theory, that EphB4 affects Kras protein stability through regulating Kras ubiquitination. We expressed Ub-Flag and Kras-myc in the 293T cells and, meanwhile, altered EphB4 levels by either knockdown or overexpression. Immunoprecipitation was performed using an anti-Myc antibody (Kras-myc) followed by immunoblot with an anti-Flag antibody (Ub-Flag). The results showed that knockdown of EphB4 by siRNA greatly increased the Kras poly-ubiquitination that coincided with decreased stability, whereas EphB4 overexpression decreased Kras ubiquitination (FIG. 19C).

It has been demonstrated that Kras poly-ubiquitination is regulated through an F-box family E3 ligase, 8-transducing repeat containing protein 1 (8-TrCP1). We confirmed that overexpressed 8-TrCP1 promoted Kras poly-ubiquitination, while knockdown of 8-TrCP1 decreased Kras ubiquitination (FIG. 19D). To gain insights into how EphB4 is involved in this process, EphB4 was overexpressed in the presence of Flag-3-TrCP1, HA-Ub and Kras-myc in 293T cells. The IP/western blot analysis showed that overexpressed EphB4 abolished 8-TrCP-mediated Kras poly-ubiquitination (FIG. 19E). The co-immunoprecipitation (Co-IP) studies further proved that the protein-protein interaction between Kras and 8-TrCP1 can be disrupted specifically by overexpressed EphB4, but not by EphB4-eGFP (an intra-cellular domain truncated form of EphB4), EphrinB2 or Her2 (FIG. 19F, upper panels). The Co-IP between EphB4 and 8-TrCP1 or the Co-IP between EphB4 and Kras showed that EphB4 competes the 8-TrCP1-Kras interaction by binding to 8-TrCP1, instead of Kras protein (FIG. 19F, middle panels).

### Example 15: Presence of a C-Terminal EphB4 Fragment Modulates 8-TrCP1 Ligase Activity Prompting Kras Monoubiquitination at the Cys118 Position

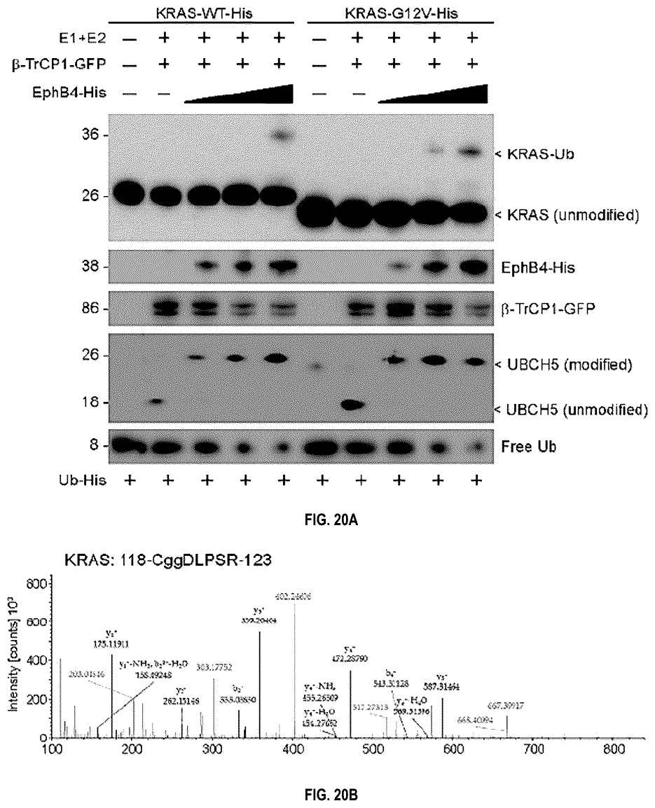

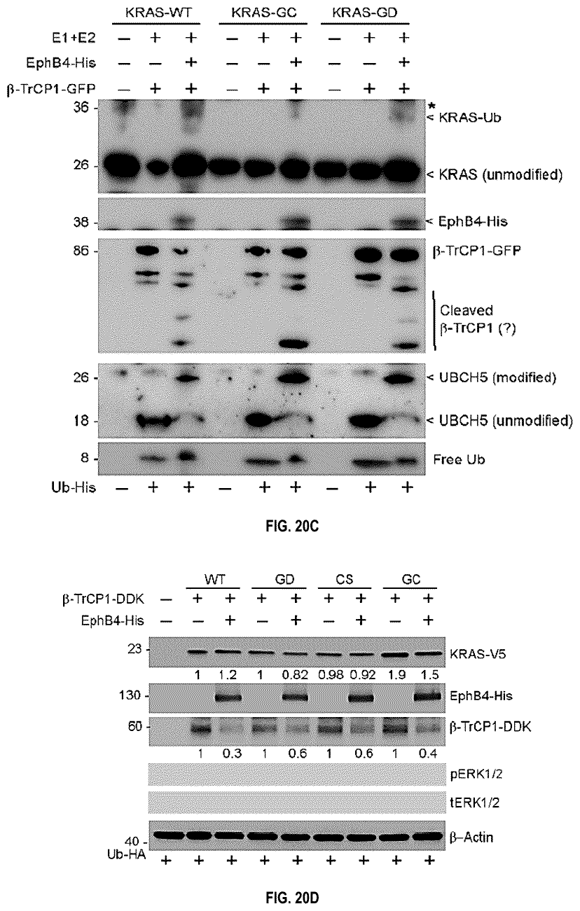

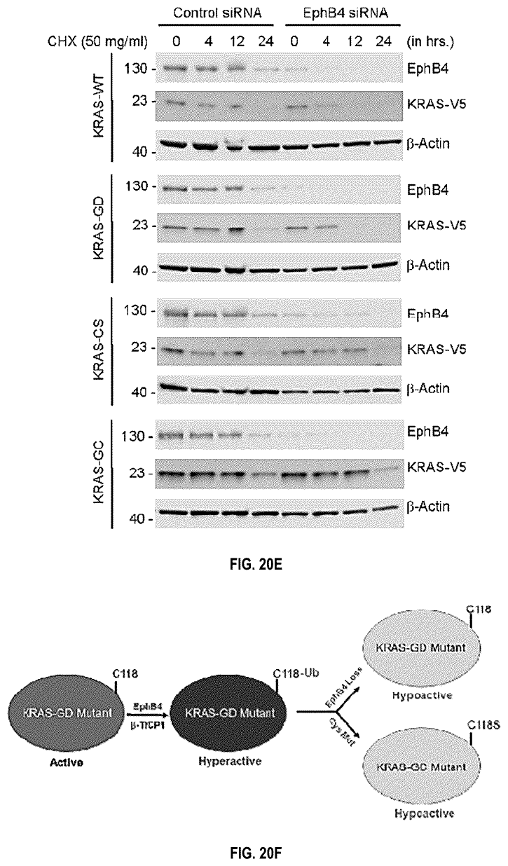

To decipher the mechanism of EphB4 inhibition of 8-TrCP1 mediated Kras polyubiquitination, we performed an in vitro ubiquitination assay. As shown in FIG. 20A, we identified a slow migrating Kras band indicative of a possible monoubiquitination only in the presence of higher concentrations of EphB4 both for the wild type (WT) and G12V mutant of Kras. We next performed mass spectrometry to identify specific attachments of a ubiquitin moiety at the cysteine 118 position (118-CggDLPSR-123) in both the WT and G12V mutant Kras (FIG. 20B). Consequently, we created Cys118 to serine (S) mutants of Kras in the G12D mutant background, named GC mutant and further performed in vitro ubiquitination and mass spectrometry analysis, as above. We identified C118 monoubiquitination both for the WT and GD mutant of Kras only in the presence of EphB4; however, the GC mutant neither showed mobility shift indicative of monoubiquitination (FIG. 20C), not did we identify any ubiquitin modified peptide following mass spectrometry analysis. Interestingly, while analyzing protein expression data, we consistently noted 8-TrCP1 fragments only in the presence of EphB4. Next, we determined steady state levels of different Kras mutants (WT, GD, CS, and GC) and found increased levels Kras GC mutant (FIG. 20D). To ascertain the role of EphB4 on Kras protein stability, we performed siRNA mediated knockdown of EphB4 and determined different Kras protein half-lives. We found loss of EphB4 negatively impacts WT, GD and CS mutants of Kras protein stability, data showed increased protein stability of the GC mutant even in the absence of EphB4 (FIG. 20E) suggesting the importance of C118 in EphB4 mediated Kras regulation. Although more stable, the GC mutant was found to be hypoactive compared to GD Kras mutant as indicated by the lower pERK1/2 level). In this study, we also noted lower 8-TrCP1 levels upon overexpression of EphB4, suggesting a negative correlation between EphB4-8-TrCP1 axis. Together, we proposed a model (FIG. 20F) where overexpression of EphB4 may lead to enhanced 8-TrCP1 mediated monoubiquitination of Kras necessary for hyperactivation of mutant Kras protein. Consequently, either the loss of EphB4 or site-specific mutation of C118 in Kras may compromise mutant Kras activity suggesting cooperativity between mutant Kras and EphB4.

### Example 16: Clinical Efficacy of sEphB4HSA targeting Ras mutant human tumors

Preclinical data indicated that EphB4-EphrinB2 pathway targeting may be efficacious in human tumors, and that all Ras mutations are subject to blocking EphB4-Ephrinb2 targeting, due to a conserved mechanism of action and critical residues conserved in all Ras forms (KRas, HRas, NRas) an all mutations within each of the Ras isoform.

We treated several patients with Ras mutations:
1. A 57 year old female with lung adenocarcinoma who has mutations in KRas12D, and concurrent mutation in ATM G2891D and PIK3CA E545K. Patient received prior radiation therapy, and failed. The tumor showed expression of EphrinB2. Patient received sEphB4-Albumin fusion protein therapy and achieved complete remission. Patient remains free of tumor for over 2 years. Patient has been off therapy for over 1 year.
2. Patient CB, 62 year old female with KRas G12C mutation, concurrent mutations of DKN2A. had adenocarcinoma of the lung. Patient had previously been treated with chemotherapy containing carboplatinum, Alimta and Avastin. Patient had short period of response lasting 5 months. Patient tumor showed EphrinB2 expression and was placed on sEphB4-albumin containing treatment with no addition of chemotherapy. Patient had response to therapy for a period of six months. Patient decided to stop therapy and eventually progressed.
3. JK. 42 year old patient with NRAS G13R mutation, concurrent GNAS, TP53 mutation has bladder cancer. Patient had cisplatinum and etoposide therapy with primary resistance. Tumor analysis showed expression of EphrinB2. Patient then received sEphB4-albumin fusion protein therapy. Patient showed tumor response lasting 4 months, with eventual progression.
4. RG. 79 year old male with NRAS mutation, concurrent mutation in CYLC L227fs, FBXWY R49Q. patient has head and neck cancer, treated with radiation therapy and EGFR antibody achieving response that lasted of 9 months. patient relapsed with tumor in the lung and the lymph nodes. Tumor biopsy showed EPhrinB2 expression. Patient was placed on sEphB4-albumin fusion protein therapy. Patient achieved complete remission and remained in remission over 2.5 months off therapy.

### SEQUENCES

The amino acid sequences listed in the accompanying sequence listing are shown using standard three letter code for amino acids.

SEQ ID NO: 1 is the amino acid sequence of human ephrin type-B receptor precursor
(NP_004435.3). Amino acid residues 1-15 encode a signal sequence.
(SEQ ID NO: 1)
MELRVLLCWASLAAALEETLLNTKLETADLKWVTFPQVDGQWEELSGLDEEQHSVRTYEVCDVQRAPGQAHWLRT
GWVPRRGAVHVYATLRFTMLECLSLPRAGRSCKETFTVFYYESDADTATALTPAWMENPYIKVDTVAAEHLTRKRPG
AEATGKVNVKTLRLGPLSKAGFYLAFQDQGACMALLSLHLFYKKCAQLTVNLTRFPETVPRELVVPVAGSCVVDAVP
APGPSPSLYCREDGQWAEQPVTGCSCAPGFEAAEGNTKCRACAQGTFKPLSGEGSCQPCPANSHSNTIGSAVCQC
RVGYFRARTDPRGAPCTTPPSAPRSWVSRLNGSSLHLEWSAPLESGGREDLTYALRCRECRPGGSCAPCGGDLTF
DPGPRDLVEPWWWVRGLRPDFTYTFEVTALNGVSSLATGPVPFEPVNVTTDREVPPAVSDIRVTRSSPSSLSLAWAV
PRAPSGAVLDYEVKYHEKGAEGPSSVRFLKTSENRAELRGLKRGASYLVQVRARSEAGYGPFGQEHHSQTQLDESE
GWREQLALIAGTAVVGVVLVLWVIVAVLCLRKQSNGREAEYSDKHGQYLIGHGTKVYIDPFTYEDPNEAVREFAKEID
VSYVKIEEVIGAGEFGEVCRGRLKAPGKKESCVAIKTLKGGYTERQRREFLSEASIMGQFEHPNIIRLEGVVTNSMPV
MILTEFMENGALDSFLRLNDGQFTVIQLVGMLRGIASGMRYLAEMSYVHRDLAARNILVNSNLVCKVSDFGLSRFLEE
NSSDPTYTSSLGGKIPIRWTAPEAIAFRKFTSASDAWSYGIVMWEVMSFGERPYWDMSNQDVINAIEQDYRLPPPPD
CPTSLHQLMLDCWQKDRNARPRFPQVVSALDKMIRNPASLKIVARENGGASHPLLDQRQPHYSAFGSVGEWLRAIK
MGRYEESFAAAGFGSFELVSQISAEDLLRIGVTLAGHQKKILASVQHMKSQAKPGTPGGTGGPAPQY
SEQ ID NO: 2 is the amino acid sequence of human serum albumin preproprotein
(NP_000468.1). Amino acid residues 25-609 encode the mature peptide.
(SEQ ID NO: 2)
MKWVTFISLLFLFSSAYSRGVFRRDAHKSEVAHRFKDLGEENFKALVLIAFAQYLQQCPFEDHVKLVNEVTEFAKTCV
ADESAENCDKSLHTLFGDKLCTVATLRETYGEMADCCAKQEPERNECFLQHKDDNPNLPRLVRPEVDVMCTAFHDN
EETFLKKYLYEIARRHPYFYAPELLFFAKRYKAAFTECCQAADKAACLLPKLDELRDEGKASSAKQRLKCASLQKFGE
RAFKAWAVARLSQRFPKAEFAEVSKLVTDLTKVHTECCHGDLLECADDRADLAKYICENQDSISSKLKECCEKPLLEK
SHCIAEVENDEMPADLPSLAADFVESKDVCKNYAEAKDVFLGMFLYEYARRHPDYSVVLLLRLAKTYETTLEKCCAAA
DPHECYAKVFDEFKPLVEEPQNLIKQNCELFEQLGEYKFQNALLVRYTKKVPQVSTPTLVEVSRNLGKVGSKCCKHP
EAKRMPCAEDYLSVVLNQLCVLHEKTPVSDRVTKCCTESLVNRRPCFSALEVDETYVPKEFNAETFTFHADICTLSEK
ERQIKKQTALVELVKHKPKATKEQLKAVMDDFAAFVEKCCKADDKETCFAEEGKKLVAASQAALGL.

### INCORPORATION BY REFERENCE

Patent documents U.S. Pat. Nos. 7,381,410; 7,862,816; 7,977,463; 8,063,183; 8,273,858; 8,975,377; 8,981,062; 9,533,026; PCT/US2020/018160; PCT/US2020/023215 and all references disclosed herein are hereby incorporated by reference in their entirety for all purposes.

### Miscellaneous

All of the articles and methods disclosed and claimed herein can be made and executed without undue experimentation in light of the present disclosure. While the articles and methods of this invention have been described in terms of embodiments, it will be apparent to those of skill in the art that variations may be applied to the articles and methods without departing from the spirit and scope of the invention. All such variations and equivalents apparent to those skilled in the art, whether now existing or later developed, are deemed to be within the spirit and scope of the invention as defined by the appended claims. All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each subject publication was specifically and subjectively indicated to be incorporated by reference in its entirety for any and all purposes. The invention illustratively described herein suitably may be practiced in the absence of any element(s) not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

1-16. (canceled)

17. A method for treating a cancer comprising administering an effective amount of a polypeptide agent that inhibits EphB4 or EphrinB2-mediated functions to a patient in need thereof, wherein the polypeptide agent is used as a first-line therapy in the treatment.

18. The method of claim 17, wherein the cancer is selected from squamous cell carcinoma of the head and neck (HNSCC), hepatocellular carcinoma (HCC), Kras mutant non-small cell lung adenocarcinoma (NSCLC), Kaposi sarcoma (KS), bladder, and cholangiocarcinoma (CCA).

19. The method of claim 18, wherein the cancer is refractory to an anticancer therapy selected from immunotherapy treatment, treatment with a chemotherapeutic agent, treatment using depleting antibodies to specific tumor antigens, treatment using agonistic, antagonistic, or blocking antibodies to co-stimulatory or co-inhibitory molecules, optionally immune checkpoint inhibitors, targeted treatment with an immunoconjugate, antibody-drug conjugate (ADC), a fusion molecule comprising a depleting antibody to specific tumor antigens tumor antigen and a cytotoxic agent, targeted treatment with a small molecule kinase inhibitor, treatment using surgery, treatment using stem cell transplantation, and treatment using radiation.

20. The method of claim 19, wherein the cancer is refractory to treatment with an immune checkpoint inhibitor.

21. The method of claim 19, wherein the cancer is refractory to treatment with radiation therapy.

22. The method of claim 19, wherein the cancer is refractory to treatment with platinum-based chemotherapy.

23. The method of claim 17, wherein the cancer comprises tumors expressing EphrinB2.

24. The method of claim 17, wherein the polypeptide agent is a ligand-binding portion of the EphB4 protein and comprises a modification that increases serum half-life.

25. The method of claim 24, wherein the polypeptide agent comprises a sequence of amino acids 1-197, 16-197, 29-197, 1-312, 16-312, 29-312, 1-321, 16-321, 29-321, 1-326, 16-326, 29-326, 1-412, 16-412, 29-412, 1-427, 16-427, 29-427, 1-429, 16-429, 29-429, 1-526, 16-526, 29-526, 1-537, 16-537 and 29-537 of SEQ ID NO: 1 (“sEphB4 polypeptide”) associated covalently or non-covalently with an albumin selected from a human serum albumin (HSA) (“sEphB4-HSA”) and bovine serum albumin (BSA) (“sEphB4-BSA”).

26. The method of claim 25, wherein the sEphB4-HSA comprises residues 16-326 of SEQ ID NO: 1 directly fused to residues 25-609 of SEQ ID NO: 2.

27. The method of claim 25, wherein the sEphB4-HSA comprises residues 16-537 of SEQ ID NO: 1 directly fused to residues 25-609 of SEQ ID NO: 2.

28. The method of claim 17, further comprising administering an anti-EGFR antibody or antibody fragment thereof.

29. The method of claim 17, further comprising administering a taxane.

30. A method for treating a cancer comprising administering an effective amount of a polypeptide agent comprising: (i) ligand-binding portion of the EphB4 protein comprising a sequence of amino acids 16-537 of SEQ ID NO: 1 and (ii) human serum albumin (HSA) comprising a sequence of amino acids 25-609 of SEQ ID NO: 2 to a patient in need thereof, wherein the polypeptide agent is used as a first-line therapy in the treatment and/or wherein the cancer is a recurrent, resistant, or refractory cancer.

31. The method of claim 30, wherein the cancer is selected from squamous cell carcinoma of the head and neck (HNSCC), hepatocellular carcinoma (HCC), Kras mutant non-small cell lung adenocarcinoma (NSCLC), Kaposi sarcoma (KS), bladder, and cholangiocarcinoma (CCA).

32. The method of claim 31 wherein the cancer is resistant or refractory to an immune checkpoint inhibitor, radiation therapy, and/or a chemotherapy.


SEQUENCE LISTING (2 sequences)

SEQ ID NO: 1 - protein, 987 aa, Artificial Sequence
  Synthetic Sequence
MELRVLLCWASLAAALEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLALIAGTAVVGVVLVLVVIVVAVLCLRKQSNGREAEYSDKHGQYLIGHGTKVYIDPFTYEDPNEAVREFAKEIDVSYVKIEEVIGAGEFGEVCRGRLKAPGKKESCVAIKTLKGGYTERQRREFLSEASIMGQFEHPNIIRLEGVVTNSMPVMILTEFMENGALDSFLRLNDGQFTVIQLVGMLRGIASGMRYLAEMSYVHRDLAARNILVNSNLVCKVSDFGLSRFLEENSSDPTYTSSLGGKIPIRWTAPEAIAFRKFTSASDAWSYGIVMWEVMSFGERPYWDMSNQDVINAIEQDYRLPPPPDCPTSLHQLMLDCWQKDRNARPRFPQVVSALDKMIRNPASLKIVARENGGASHPLLDQRQPHYSAFGSVGEWLRAIKMGRYEESFAAAGFGSFELVSQISAEDLLRIGVTLAGHQKKILASVQHMKSQAKPGTPGGTGGPAPQY

SEQ ID NO: 2 - protein, 609 aa, Artificial Sequence [sequence omitted]